SEQUENCE LISTING (4 sequences)

SEQ ID NO: 1 - RNA, 19 nt, Artificial Sequence
  Synthetic anti-GAPDH silencer RNA (siRNA)
ggucauccaugacaacuuu

SEQ ID NO: 2 - RNA, 19 nt, Artificial Sequence
  Synthetic anti-GAPDH silencer RNA (siRNA)
aaaguugucauggaugacc

SEQ ID NO: 3 - DNA, 20 bp, Artificial Sequence
  Synthetic oligonucleotide
aguacugcuuacgauacgtt

SEQ ID NO: 4 - DNA, 21 bp, Artificial Sequence
  Synthetic oligonucleotide
ccguaucguaagcaguacutt

(19) United States
(12) Patent Application Publication
(10) Pub. No.: US 2013/0165734 A1
(43) Pub. Date: Jun. 27, 2013

(54) TIME-DOMAIN TRANSDUCTION SIGNALS AND METHODS OF THEIR PRODUCTION AND USE

(71) Applicant: Nativis, Inc., Seattle, WA (US)

(72) Inventor: John T. Butters, Langley, WA (US)

(73) Assignee: Nativis, Inc., Seattle, WA (US)

(21) Appl. No.: 13/662,164

(22) Filed: Oct. 26, 2012

Related U.S. Application Data
(63) Continuation of application No. 13/263,318.

(51) Int. Cl. A61N 2/00; G01N 27/00

(52) U.S. Cl. CPC A61N 2/002; G01N 27/00
USPC 600/13; 324/228

(57) ABSTRACT

A storage medium having a low frequency time-domain signal stored thereon, and methods of generating, scoring, testing and using the signals are disclosed. In one general embodiment, the signal is derived from a taxane-like compound or an siRNA against human GADPH, and is useful in treating cancer in a subject by exposing the subject a low-magnetic field transduction of the signal. Also disclosed are improved signal transduction methods.

### FIELD OF THE INVENTION

The present invention relates to a storage medium having a low-frequency time-domain signal stored thereon, and methods for generating, scoring, testing and using the signal.

### BACKGROUND OF THE INVENTION

One of the accepted paradigms in the fields of chemistry and biochemistry is that chemical or biochemical effector agents, e.g., molecules, interact with target biological systems through various physicochemical forces, such as ionic, charge, or dispersion forces or through the cleavage or formation of covalent or charge-induced bonds. These forces presumably involve field effects, e.g., electrostatic and magnetic field effects, by which the presence of the effector influences the condition or response of the target.

One question raised by this paradigm is whether interactions between effector and target require the presence of the effector itself or whether at least some critical effector-target interactions can be achieved by simulating field effects associated with effector molecules with signals derived from the effector molecules. Studies undertaken to examine the interaction between effector-molecule signals and biological targets were reported in co-owned PCT applications WO 2006/073491 A2 and WO 2008/063654 A2, both of which are incorporated by reference herein. These applications describe studies in which low-frequency time-domain signals recorded for a number of bio-active compounds (effectors), in accordance with apparatus and methods detailed in the applications, were used in induce compound-specific effects in biological target systems.

PCT application WO 2006/073491, published Jul. 13, 2006 discloses studies in which (a) low-frequency time-domain signals recorded for L(+) arabinose were shown to induce the araC-PBAD bacterial operon, as discussed on pages 47-50 of the application, with respect to FIGS. 30C-30F; (b) low-frequency signals recorded for glyphosphate, the active ingredient in a well-known herbicide, were shown to substantially inhibit stem growth in pea sprouts, as discussed on pages 50-51 of the application, with respect to FIGS. 31 and 32A and 32B; (c) low-frequency signals recorded for gibberellic acid, a plant hormone, were shown to significantly increase average stem length in live sugar peat sprouts, as discussed on pages 51-53 of the application, with respect to FIG. 33; and (d) low-frequency signals recorded for phepropeptin, a proteasome inhibitor, were shown to decrease the activity of the 20S proteosome enzyme, as discussed on pages 53-54 of the application, with respect to FIG. 34.

WO 2008/063654 A2, published May 9, 2008, details a number of studies in which low-frequency time-domain signals for the anti-tumor compound taxol, generated in accordance with methods disclosed herein were shown to stimulate and stabilize tubulin assembly in an in vitro tubulin polymerization assay, as described on pages 42-45 of the PCT application, with respect to data shown in FIGS. 16A-16E. Additional studies reported in the application, on pages 45-46 of the application, demonstrate that the same signals are effective in reducing tumor growth in animals injected with glioblastoma cells.

Among the findings from the studies described above is that the ability of agent-specific, time-domain signals to transduce (affect) a biochemical or biological target system can be optimized by a number of strategies. One of these strategies involves scoring recorded time-domain signals by one or more scoring algorithms to identify those signals that contain the highest spectral information. This scoring is used to screen recorded time-domain signals for those that are most likely to give a strong transduction effect. An improvement in this strategy is to record time-domain signals at each of a number of different magnetic-signal injection conditions, by injecting different levels of white noise or DC offset during recording, and scoring the resulting signals for highest spectral information. These strategies are detailed in both of the above-cited PCT applications.

A third strategy, disclosed in the '654 application, is designed particularly for applications in which a recorded time-domain signal is intended for transducing an animal system, for example, for treating a disease condition in a subject. The strategy involves screening time-domain signals for their ability to effectively transduce an in vitro target system that includes at least some of the critical biological response components of the animal system. For example, time-domain signals recorded for paclitaxol at each of a number of different magnetic-signal inputs can be initially scored for spectral information, and those signals with the highest score are then further screened for their ability to promote tubulin polymerization in an in vitro tubulin assay. Those signals showing the highest transduction effect in the in vitro system are then selected for use in transducing an animal target. This strategy has the advantage that a large number of candidate signals can be easily screened for actual transduction effect, to identify optimal transducing signals. The strategy is preferably combined with one or both of the above signal-scoring methods, using the highest-scoring signals as candidates for the in vitro transduction screening.

The present application is directed to strategies for generating, scoring, and selecting time-domain signals that can be characterized by a drug-related mechanism of action observed when a biochemical or biological system is exposed to a magnetic field produced by the signals, and to improved apparatus and methods for transducing such as system.

### SUMMARY OF THE INVENTION

The invention includes, in one aspect, a tangible data storage medium having stored thereon, a low-frequency time domain signal effective to produce a magnetic field capable, as a mechanism if action, of stabilizing microtubule formation in an in vitro tubulin assay containing a suspension of tubulin, where the signal is supplied to electromagnetic transduction coil(s) at a signal current calculated to produce a magnetic field strength within a range between 10⁻⁴ to 10⁻¹¹ Tesla, e.g., 10⁻⁸ to 10⁻¹¹ Tesla, and where the degree of stabilization of microtubule formation in the assay produced in the presence of the magnetic field is substantially greater than that observed in the absence of the field.

The signal carried on the storage medium may be produced by the steps of: (a) placing in a sample container having both magnetic and electromagnetic shielding, a sample of a taxane-like compound known to stabilize microtubule formation in such a tubulin sample, wherein the sample acts as a signal source for low-frequency molecular signals; and wherein the magnetic shielding is external to a cryogenic container; (b) recording a plurality of low-frequency, time-domain signals composed of sample source radiation in the cryogenic container, (c) scoring the signals produced in step (b) by one of (i) the peak areas values above a predetermined value as determined from an enhanced autocorrelation of the signal, and (ii) a histogram of the power spectrum of the signal, determined by spectral analysis, and (d) identifying from among the signals having the highest score or scores from step (c) one or more signals that are effective in stabilizing microtubule formation in an in vitro tubulin assay, when the tubulin sample is exposed to a magnetic field produced by supplying the signal to electromagnetic transducer coil(s) at a signal current calculated to produce a magnetic field strength in the range between 10⁻⁴ to 10⁻¹¹ Tesla.

Step (b) in producing the signal may be carried out by recording a plurality of low-frequency, time-domain signals composed of sample source radiation in the cryogenic container includes recording the signals at each of a plurality of different stimulus magnetic field conditions selected from the group consisting of: (i) white noise, injected at voltage level calculated to produce a selected-strength magnetic field at the sample of between 0 and 1 G (Gauss), and/or (ii) a DC offset, injected at voltage level calculated to produce a selected-strength magnetic field at the sample of between 0 and 1 G.

Step (a) in producing the signal may be carried out by preparing a taxane-like sample in an aqueous medium having a physiological salt concentration. The taxane-like compound may be a taxane compound, such as taxol (paclitaxol).

In one embodiment, the ability of the time-domain signal to stabilize microtubule formation in the in vitro tubulin assay is assessed by supplying the signal to electromagnetic transducer coil(s) at a signal current calculated to produce an incremented magnetic field which is cycled within a range between 10⁻⁴ to 10⁻¹¹ Tesla, e.g., 10⁻⁸ to 10⁻¹¹ Tesla.

In another aspect, the invention includes a tangible data storage medium having stored thereon, a low-frequency time domain signal effective to produce a magnetic field capable, as a mechanism of action, of inhibiting mRNA expression of a selected gene in an in vitro assay containing cultured cells, where the signal is supplied to electromagnetic transduction coil(s) at a signal current calculated to produce a magnetic field strength within a range between 10⁻⁴ to 10⁻¹¹ Tesla, and where the degree of inhibition of mRNA expression in the assay in the presence of the magnetic field is substantially greater than that observed in the absence of the field.

The signal carried on the storage medium may be produced by the steps of: (a) placing in a sample container having both magnetic and electromagnetic shielding, a sample of an siRNA compound known to mRNA expression of a selected gene in an in vitro assay in which the cultured cells are exposed to the compound, wherein the sample acts as a signal source for low-frequency molecular signals; and wherein the magnetic shielding is external to a cryogenic container; (b) recording a plurality of low-frequency, time-domain signals composed of sample source radiation in the cryogenic container; (c) scoring the signals produced in step (b) by one of (i) the peak areas values above a predetermined value as determined from an enhanced autocorrelation of the signal, and (ii) a histogram of the power spectrum of the signal, determined by spectral analysis, and (d) identifying from among the signals having the highest score or scores from step (c) one or more signals that are most effective in inhibiting the mRNA expression of the selected gene, when an in vitro assay containing cultured is exposed to a magnetic field produced by supplying the signal to electromagnetic transducer coil(s) at a signal current calculated to produce a magnetic field within a range between 10⁻⁴ to 10⁻¹¹ Tesla, e.g., 10⁻⁸ to 10⁻¹¹ Tesla.

Step (b) in producing the signal may be carried out by recording a plurality of low-frequency, time-domain signals composed of sample source radiation in the cryogenic container includes recording the signals at each of a plurality of different stimulus magnetic field conditions selected from the group consisting of: (i) white noise, injected at voltage level calculated to produce a selected-strength magnetic field at the sample of between 0 and 1 G (Gauss), and/or (ii) a DC offset, injected at voltage level calculated to produce a selected-strength magnetic field at the sample of between 0 and 1 G.

Step (a)) used in producing the signal may include preparing an anti-GADPH siRNA sample in an aqueous medium having a physiological salt concentration. The anti-GADPH may be a double-stranded RNA having the sequence identified by SEQ ID NO: 1.

In one embodiment, the ability of the time-domain signal to inhibit expression of GADPH mRNA expression in the in vitro assay is assessed by supplying the signal to electromagnetic transducer coil(s) at a signal current calculated to produce an incremented magnetic field which is cycled within a range between 10⁻⁴ to 10⁻¹¹ Tesla, e.g., e.g., 10⁻⁸ to 10⁻¹¹ Tesla.

In still another aspect, the invention is directed to an improvement in a method for producing an agent-like response in a system, by exposing the system to a magnetic field produced by one or more electromagnetic transducer coils to which is supplied a selected low-frequency time-domain signal over a given exposure period. The improvement includes adjusting the magnetic field to which the subject is exposed by applying to the transducer coils, a signal current calculated to produce a magnetic field within a range between 10⁻⁴ to 10⁻¹¹ Tesla, e.g., 10⁻⁸ to 10⁻¹¹ Tesla, where the magnetic field supplied to the subject is supplied in cycles of increasing-field increments, over a selected signal-current range, where each signal-current increment in a cycle is applied in defined-duration pulses over the known given period.

For use in treating a subject having a tumor whose cells are inhibited in the presence of taxol, the magnetic field to which the subject is exposed may be produced by supplying to the one or more electromagnetic transduction coils, a low-frequency time domain signal effective to stabilize microtubule formation in an in vitro tubulin assay containing a suspension of tubulin, in the absence of added compound, to a magnetic field produced by supplying the signal to electromagnetic transducer coil(s) at a signal current calculated to produce a magnetic field in the range between 10⁻⁴ to 10⁻¹¹ Tesla, relative to degree of microtubule stabilization observed in the absence of the supplied signal.

The low-frequency time-domain signal may be produced by the steps of: (a) placing in a sample container having both magnetic and electromagnetic shielding, a sample of a taxane-like compound known to stabilize microtubule formation in such a tubulin sample, wherein the sample acts as a signal source for low-frequency molecular signals; and wherein the magnetic shielding is external to a cryogenic container; (b) recording a plurality of low-frequency, time-domain signals composed of sample source radiation in the cryogenic container, (c) scoring the signals produced in step (b) by one of (i) the peak areas values above a predetermined value as determined from an enhanced autocorrelation of the signal, and (ii) a histogram of the power spectrum of the signal, determined by spectral analysis, and (d) identifying from among the signals having the highest score or scores from step (c) one or more signals that are most effective in stabilizing microtubule formation in an in vitro tubulin assay, when the tubulin sample is exposed to a magnetic field produced by supplying the signal to electromagnetic transducer coil(s) at a signal current calculated to produce a magnetic field within a range in the range between 10⁻⁴ to 10⁻¹¹ Tesla, e.g., 10⁻⁸ to 10⁻¹¹ Tesla.

The subject may be exposed to the magnetic field, either continuously or on a daily basis, at least over a three-week treatment period, and the method further includes measuring changes in the size of the tumor over the treatment period.

For use in treating in a subject whose cells are inhibited by the presence of an anti-GADPG siRNA compound, the magnetic field to which the subject is exposed may be produced by supplying to the one or more electromagnetic transduction coils, a low-frequency time domain signal effective to produce an siRNA-specific inhibition of GADPH protein or GADPH mRNA, relative to that observed for a signal derived under identical conditions from a scrambled-sequence siRNA control, in an in vitro siRNA assay in which 549 lung carcinoma cells are exposed to magnetic field produced by supplying the signal to electromagnetic transducer coil(s) at a signal current calculated to produce a selected-strength magnetic field in the range between 10⁻⁴ to 10⁻¹¹ Tesla.

The time-domain signal may be produced by the steps of: (a) placing in a sample container having both magnetic and electromagnetic shielding, a sample of an siRNA compound known to inhibit GADPH protein or GADPH mRNA expression in an in vitro assay in which 549 lung carcinoma cells are exposed to the compound, wherein the sample acts as a signal source for low-frequency molecular signals; and wherein the magnetic shielding is external to a cryogenic container; (b) recording a plurality of low-frequency, time-domain signals composed of sample source radiation in the cryogenic container, (c) scoring the signals produced in step (b) by one of (i) the peak areas values above a predetermined value as determined from an enhanced autocorrelation of the signal, and (ii) a histogram of the power spectrum of the signal, determined by spectral analysis, and (d) identifying from among the signals having the highest score or scores from step (c) one or more signals that are most effective in inhibiting GADPH protein and GADPH mRNA expression, when an in vitro assay containing 549 lung carcinoma cells when the cells are exposed to a magnetic field produced by supplying the signal to electromagnetic transducer coil(s) at a signal current calculated to produce a magnetic field within a range between 10⁻⁴ to 10⁻¹¹ Tesla, e.g., 10⁻⁸ to 10⁻¹¹ Tesla.

Also disclosed is a method of treating a subject having a condition that is responsive to a therapeutic agent capable of producing an observable agent-specific effect in an in vitro cell-culture or cell-free system, comprising the steps of:

(a) placing the subject system within the interior region of one or more electromagnetic transducer coils,

(b) supplying to the transducer coils, a low-frequency time-domain signal to produce a magnetic field that is effective, when supplied to the in vitro system under identical conditions, to produce the agent-specific effect, at a signal current calculated to produce a selected-strength magnetic field at the coils in a range between 10⁻⁴ to 10⁻¹¹ Tesla, e.g., 10⁻⁸ to 10⁻¹¹ Tesla,

(c) exposing the subject to the magnetic field produced in step (b), over a time period sufficient to produce a measurable agent-specific response in the subject.

For use in treating in a condition of the CNS that would be responsive to the therapeutic agent, but for the presence of the blood brain barrier, the exposing step (c) includes exposing the region of the CNS having such condition to the magnetic field. For example, in treating a subject having a CNS tumor whose cells are inhibited in the presence of taxol, and the low-frequency signal to which the subject is exposed is effective to produce a taxol-specific polymerization response in an in vitro tubulin assay, when a tubulin sample is exposed to a magnetic field produced by supplying the signal to electromagnetic transducer coil(s) at a signal current calculated to produce a selected-strength magnetic field within a range between 10⁻⁴ to 10⁻¹⁴ Tesla, e.g., 10⁻⁸ to 10⁻¹¹ Tesla.

These and other objects and features of the invention will be more fully understood when the following detailed description of the invention is read in conjunction with the accompanying drawings.

### BRIEF DESCRIPTION OF THE DRAWINGS

FIG. 1 is a diagram of a signal-detection apparatus constructed in accordance to one embodiment of the invention;

FIG. 2 is a diagram showing components the signal-processing apparatus of FIG. 1;

FIG. 3 is a flow diagram of the signal detection and processing performed by an embodiment of the present system;

FIG. 4 shows a high-level flow diagram of data flow for processing time-domain signals in accordance with an embodiment of the invention;

FIG. 5 is a flow diagram of an histogram-bin algorithm in accordance with one scoring algorithm that can be used in the invention;

FIG. 6 is a flow diagram of a power spectral density algorithm in accordance with another algorithm that can be used in the invention;

FIGS. 7A-7C illustrate transduction configurations used for detecting signal-induced changes in tubulin polymerization (7A), for detecting signal-induced changes in cultured cells (7B) and for detecting signal-induced changes in an animal (7C);

FIGS. 8A-8C show components in a transduction apparatus designed for (i) transducing a cell-based or animal system (8A) and showing a schematic diagram of an attenuator unit in the apparatus (8B), and (ii) for transducing an in vitro non-cell based system (8C);

FIGS. 9A and 9B are plots showing increase in light scattering as an indicator of increase in tubulin polymerization, in a buffer control (lower trace), tubulin control (middle trace), and tubulin plus 4 μM paclitaxel (upper trace) (9A), and the same traces but where the upper trace represents tubulin transduced with a paclitaxol signal (9B);

FIGS. 10A-10F are photomicrographs of tubulin samples, taken at both 21,000× and 52,000× for untreated controls (10A and 10B), tubulin in the presence of cremophore (10C and 10D), and tubulin exposed to white noise (10E and 10F), all after 10 minutes incubation;

FIGS. 11A-11D are photomicrographs of tubulin samples, taken at both 21,000× and 52,000× after 10 minute exposure to 4 μM paclitaxel for 10 minutes;

FIGS. 12A-12D are photomicrographs of tubulin samples, taken at both 21,000× and 52,000× after 10 minute exposure by transduction by a paclitaxel time-domain signal for 10 minutes, in accordance with the invention;

FIGS. 13A-13D are photomicrographs of 549 lung carcinoma cells in culture in the absence and presence of added taxol (FIGS. 13A, 13B, respectively), and in the absence and presence of a taxol time-domain signal (FIGS. 13C, 13D, respectively);

14A and 14B show a time-domain signal for paclitaxel over a 60-second interval (14A) and a power-spectral-density estimate for the signal (14B);

FIGS. 15A and 15 B show the in GAPDH protein (15A) and GAPDH mRNA (15B in A549 cells exposed over a 48 hr to 72 hr period to an siRNA time domain signal, in accordance with the invention.

FIGS. 16A and 16B show a time-domain signal for siRNA against GADPH protein over a 60-second interval (16A) and a power-spectral-density estimate for the signal (16B); and

FIG. 17 is a plot of normalized tumor volume, relative to control, averaged for Taxol Signal M23 Treated mice, after exposure by transduction to a paclitaxel time-domain signal for over a 23 day period in accordance with the invention.

### DETAILED DESCRIPTION OF THE INVENTION

### I. Definitions

The terms below have the following definitions unless indicated otherwise.

“Magnetic shielding” refers to shielding that decreases, inhibits or prevents passage of magnetic flux as a result of the magnetic permeability of the shielding material.

“Electromagnetic shielding” refers to, e.g., standard Faraday electromagnetic shielding, or other methods to reduce passage of electromagnetic radiation.

“Time-domain signal” or “time-series signal” refers to a signal with transient signal properties that change over time.

“Low-frequency” refers to a frequency range from DC to about 50 kHz. A low-frequency time domain signal is one having its major frequency components in the 0-50 kHz range, typically 0-20 kHz range.

“Sample-source radiation” refers to magnetic flux or electromagnetic flux emissions resulting from molecular motion of a sample, or electromagnetic fields produced by short-range or long-range interactions between two of more molecules undergoing molecular motion. Because sample source radiation is produced in the presence of an injected magnetic-field stimulus,” it is also referred to as “sample source radiation superimposed on injected magnetic field stimulus.”

“Stimulus magnetic field” or “Magnetic-field stimulus” refers to a magnetic field produced by injecting (applying) to magnetic coils surrounding a sample, one of a number of electromagnetic signals that may include (i) white noise, injected at voltage level calculated to produce a selected magnetic field at the sample of between 0 and 1 G (Gauss), (ii) a DC offset, injected at voltage level calculated to produce a selected magnetic field at the sample of between 0 and 1 G, and (iii) a combination of (i) and (ii). The injected noise and/or offset may be varied incrementally and systematically, for generating a plurality of time-domain signals at different magnetic-filed conditions.

The “magnetic field strength” produced at the sample, by supplying a time domain signal to transduction coils, may be readily calculated using known electromagnetic relationships, knowing the shape and number of windings in the injection coil, the voltage applied to coils, and the distance between the injection coils and the sample, according to known methods as described below.

A “selected stimulus magnetic-field condition” refers to a selected voltage applied to a white noise or DC offset signal, or a selected sweep range, sweep frequency and voltage of an applied sweep stimulus magnetic field.

“White noise” means random noise or a signal having simultaneous multiple frequencies, e.g. white random noise or deterministic noise. “Gaussian white noise” means white noise having a Gaussian power distribution. “Stationary Gaussian white noise” means random Gaussian white noise that has no predictable future components. “Structured noise” is white noise that may contain a logarithmic characteristic which shifts energy from one region of the spectrum to another, or it may be designed to provide a random time element while the amplitude remains constant. These two represent pink and uniform noise, as compared to truly random noise which has no predictable future component. “Uniform noise” means white noise having a rectangular distribution rather than a Gaussian distribution.

“Frequency-domain spectrum” refers to a Fourier frequency plot of a time-domain signal.

“Spectral components” refer to singular or repeating qualities within a time-domain signal that can be measured in the frequency, amplitude, and/or phase domains. Spectral components will typically refer to signals present in the frequency domain.

“Faraday cage” refers to an electromagnetic shielding configuration that provides an electrical path to ground for unwanted electromagnetic radiation, thereby quieting an electromagnetic environment.

A “signal-analysis score” refers to a score based on analysis of a time-domain signals by one of the scoring algorithms discussed below.

An “optimized agent-specific time-domain signal” refers to a time-domain signal having a maximum or near-maximum signal-analysis score.

“In vitro system” refers to a biochemical system having of one or more biochemical components, such as nucleic acid or protein components, including receptors and structural proteins isolated or derived from a virus, bacteria, or multicellular plant or animal. An in vitro system typically is a solution or suspension of one or more isolated or partially isolated in vitro components in an aqueous medium, such as a physiological buffer. The term also refers to a cell culture system containing bacterial or eukaryotic cells in a culture medium.

“Mammalian system” refers to a mammal, include a laboratory animal such as mouse, rat, or primate that may serve as a model for a human disease, or a human patient.

“Agent-specific effect” refers to an effect observed when an in vitro or mammalian system is exposed to a chemical or biochemical agent (effector). Examples of agent-specific in vitro effects include, for example, a change in the state of aggregation of components of the system, the binding the an agent to a target, such as a receptor, and the change in growth or division of cells in culture.

“Mechanism of action” refers to a mechanism by which an effector achieves its effect on a biochemical or biological system, where the same mechanism of action is the same or presumed to be the same both in vitro and in vivo systems. Where an agent acts through more than one mechanism of action, as is characteristic of many pharmaceutical compounds do in vivo, the agent's mechanism of action refers to an identified mechanism that can be demonstrated in a simplified in vitro system and demonstrated or presumed to operate in an in vivo animal system. For example, a taxane compound, e.g., taxol, has as one mechanism of action, stabilizing microtubule formation by tubulin polymerization in an in vitro tubulin system, by stabilizing GDP-bound tubulin in microtubule, and is presumed to operate through the same mechanism in inhibiting cell division in a treated animal, even though taxanes are known to have other mechanisms of action and biological effects in an animal system.

A “taxane compound” refers a class of diterpine compounds produced by the plants of the genus Taxus, and chemical analogs thereof, including but not limited to taxol (paclitaxel), docetaxel, larotaxel, ortataxel and tesetaxel.

A “taxane-like compound” refers to compounds that operate through a mechanism of action involving stabilization of microtubule formation from tubulin. Included in this definition are taxane compounds and epithilones, such as epothilones A to F, and analogs thereof, such as ixabepilone (epithilone B). These compounds are known to bind to the αβ-tubulin heterodimer subunit, like taxanes, and once bound, decrease the rate of dissociation of the heterodimers. Epothilone B has also been shown to induce tubulin polymerization into microtubules without the presence of GTP. This is caused by formation of microtubule bundles throughout the cytoplasm. Finally, epothilone B also causes cell cycle arrest at the G2-M transition phase, thus leading to cytotoxicity and eventually cell apoptosis. (Balog, D. M.; Meng, D.; Kamanecka, T.; Bertinato, P.; Su, D.-S.; Sorensen, E. J.; Danishefsky, S. J. Angew. Chem. 1996, 108, 2976. Some endotoxin-like properties known from paclitaxel, however, like activation of macrophages synthesizing inflammatory cytokines and nitric oxide, are not observed for epothilone B.

An “siRNA compound” refers to a double-stranded RNA molecule, typically about 20-25 bases long, that has sequence specificity for one or more genes. One mechanism of action of an siRNA involves the RNA interference (RNAi) pathway, with siRNA inducing a gene-specific RNAi which then interferes with transcription of the gene in producing mRNA.

A “selected magnetic field strength within q range between 10⁻⁴ to 10⁻¹¹ Tesla” refers to the magnetic field strength produced by one or more transduction coils to which is applied a time-domain signal current calculated to produce a magnetic field strength that is either a selected constant field strength between 10⁻⁴ to 10⁻¹¹ Tesla, or the magnetic field produced by a series of signal currents calculated to produce a plurality of incremental field strengths within a selected range, at least a portion of which is within the range 10⁻⁴ to 10⁻¹¹ Tesla, and preferably within the range 10⁻⁸ to 10⁻¹¹ Tesla, e.g., 10⁻⁸ to 10⁻¹¹ Tesla. “Transduction” as applied herein refers to changes produced in a biochemical or biological system in response to a magnetic field produced and selected for its ability to affect the system through a given mechanism of action.

### II. Recording Apparatus and Method

The recording apparatus for producing time-domain signals from samples of a selected agent is detailed in co-owned PCT application WO2008/063654, which is incorporated herein Certain preferred embodiments of the apparatus and scoring algorithms are described below.

The apparatus is used by placing a sample within the magnetically shielded faraday cage in close proximity to the coil that generates the stimulus signal and the gradiometer that measures the response. A stimulus signal is injected through the stimulus coil, and this signal may be modulated until a desired optimized signal is produced. The molecular electromagnetic response signal, shielded from external interference by the faraday cage and the field generated by the stimulus coil, is then detected and measured by the gradiometer and SQUID. The signal is then amplified and transmitted to any appropriate recording or measuring equipment.

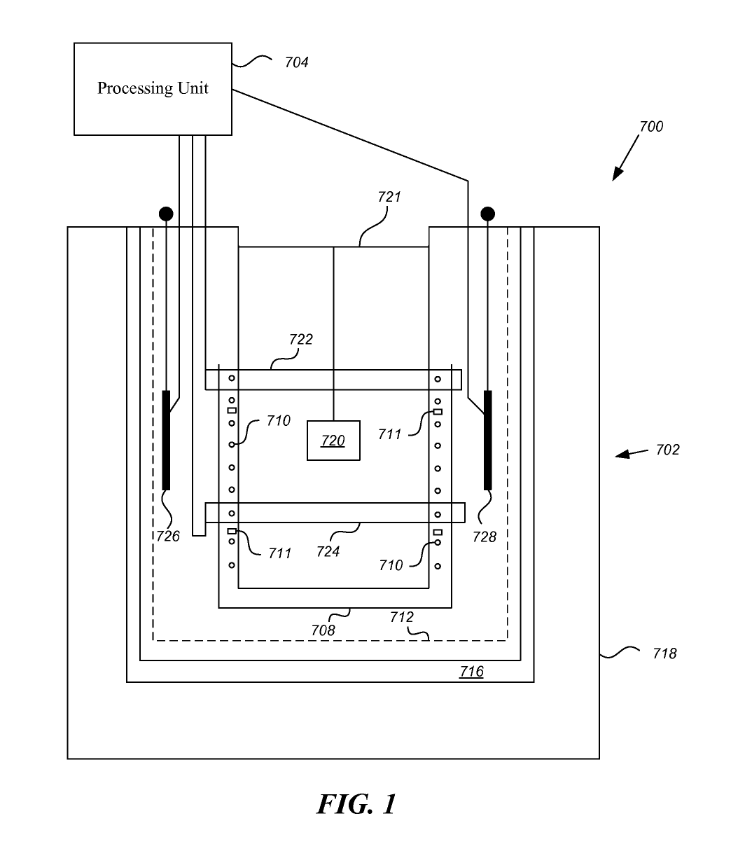

FIG. 1 shows one embodiment of an apparatus for electromagnetic emission detection and a processing system. Apparatus 700 includes a detection unit 702 coupled to a processing unit 704. Although the processing unit 704 is shown external to the detection unit 702, at least a part of the processing unit can be located within the detection unit.

The detection unit 702, which is shown in a cross-sectional view in FIG. 1, includes multiple components nested or concentric with each other. A sample chamber or faraday cage 706 is nested within a metal cage 708. Each of the sample chamber 706 and the metal cage 708 can be comprised of aluminum material. The sample chamber 706 can be maintained in a vacuum and may be temperature controlled to a preset temperature. The metal cage 708 is configured to function as a low pass filter.

Between the sample chamber 706 and the metal cage 708 and encircling the sample chamber 706 are a set of parallel heating coils or elements 710. One or more temperature sensor 711 is also located proximate to the heating elements 710 and the sample chamber 706. For example, four temperature sensors may be positioned at different locations around the exterior of the sample chamber 706. The heating elements 710 and the temperature sensor(s) 711 may be configured to maintain a certain temperature inside the sample chamber 706.

A shield 712 encircles the metal cage 708. The shield 712 is configured to provide additional magnetic field shielding or isolation for the sample chamber 706. The shield 712 can be comprised of lead or other magnetic shielding materials. The shield 712 is optional when sufficient shielding is provided by the sample chamber 706 and/or the metal cage 708.

Surrounding the shield 712 is a cryogen layer 716 with G10 insulation. The cryogen may be liquid helium. The cryogen layer 716 (also referred to as a cryogenic Dewar) is at an operating temperature of 4 degrees Kelvin. Surrounding the cryogen layer 716 is an outer shield 718. The outer shield 718 is comprised of nickel alloy and is configured to be a magnetic shield. The total amount of magnetic shielding provided by the detection unit 702 is approximately −100 dB, −100 dB, and −120 dB along the three orthogonal planes of a Cartesian coordinate system.

The various elements described above are electrically isolated from each other by air gaps or dielectric barriers (not shown). It should also be understood that the elements are not shown to scale relative to each other for ease of description.

A sample holder 720 can be manually or mechanically positioned within the sample chamber 706. The sample holder 720 may be lowered, raised, or removed from the top of the sample chamber 706. The sample holder 720 is comprised of a material that will not introduce Eddy currents and exhibits little or no inherent molecular rotation. As an example, the sample holder 720 can be comprised of high quality glass or Pyrex.

The detection unit 702 is configured to handle solid, liquid, or gas samples. Various sample holders may be utilized in the detection unit 702. For example, depending on the size of the sample, a larger sample holder may be utilized. As another example, when the sample is reactive to air, the sample holder can be configured to encapsulate or form an airtight seal around the sample. In still another example, when the sample is in a gaseous state, the sample can be introduced inside the sample chamber 706 without the sample holder 720. For such samples, the sample chamber 706 is held at a vacuum. A vacuum seal 721 at the top of the sample chamber 706 aids in maintaining a vacuum and/or accommodating the sample holder 720.

A sense coil 722 and a sense coil 724, also referred to as detection coils, are provided above and below the sample holder 720, respectively. The coil windings of the sense coils 722, 724 are configured to operate in the direct current (DC) to approximately 50 kilohertz (kHz) range, with a center frequency of 25 kHz and a self-resonant frequency of 8.8 MHz. The sense coils 722, 724 are in the second derivative form and are configured to achieve approximately 100% coupling. In one embodiment, the coils 722, 724 are generally rectangular in shape and are held in place by G10 fasteners. The coils 722, 724 function as a second derivative gradiometer.

Helmholtz coils 726 and 728 may be vertically positioned between the shield 712 and the metal cage 708, as explained herein. Each of the coils 726 and 728 may be raised or lowered independently of each other. The coils 726 and 728, also referred to as magnetic-field stimulus generation coils, are at room or ambient temperature. The noise generated by the coils 726, 728 is approximately 0.10 Gauss.

The degree of coupling between the emissions from the sample and the coils 722, 724 may be changed by repositioning the sample holder 720 relative to the coils 722, 724, or by repositioning one or both of the coils 726, 728 relative to the sample holder 720.

The processing unit 704 is electrically coupled to the coils 722, 724, 726, and 728. The processing unit 704 specifies the magnetic-field stimulus, e.g., Gaussian white noise stimulus to be injected by the coils 726, 728 to the sample. The processing unit 104 also receives the induced voltage at the coils 722, 724 from the sample's electromagnetic emissions mixed with the injected magnetic-field stimulus.

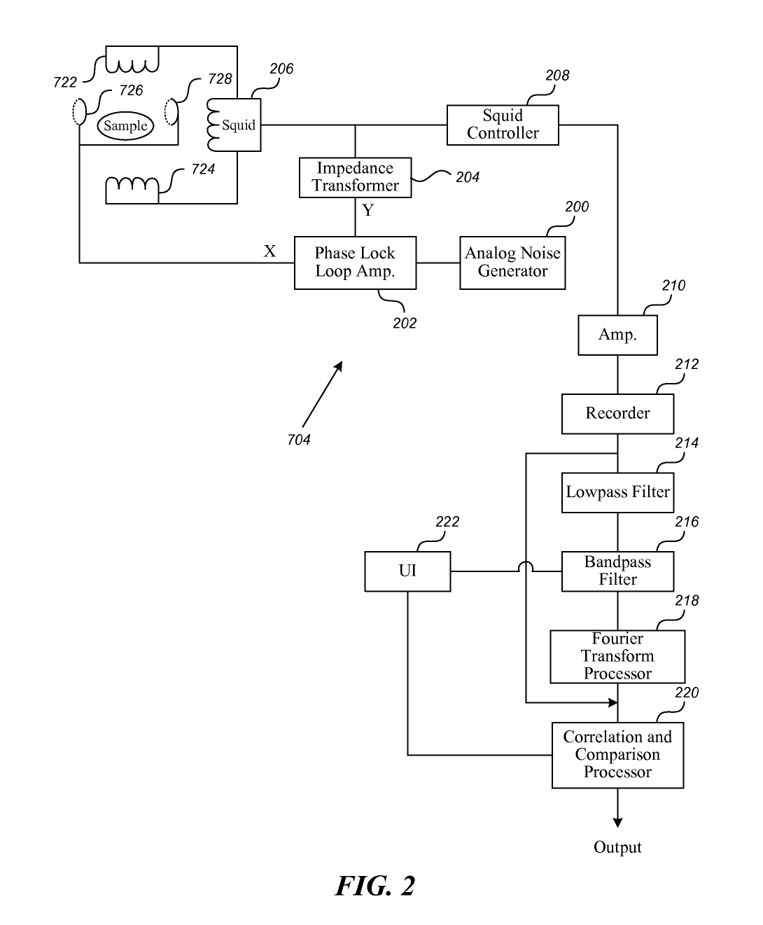

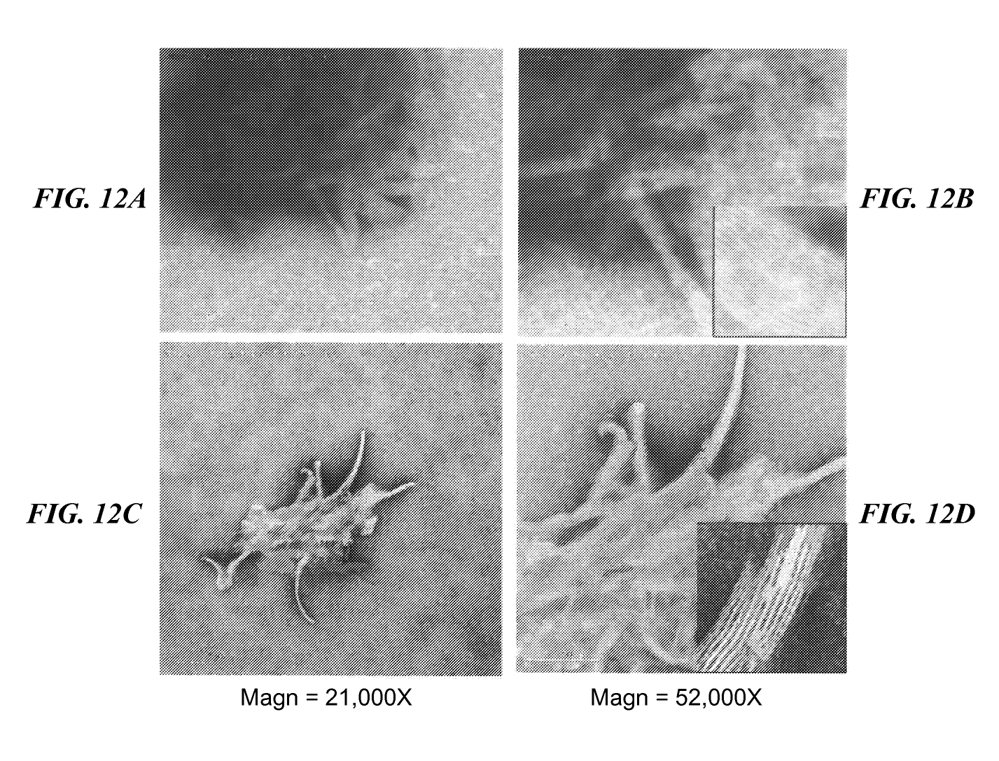

FIG. 2 is a block diagram of the processing unit shown at 704 in FIG. 12. A dual phase lock-in amplifier 202 is configured to provide a first magnetic-field signal (e.g., “x” or noise stimulus signal) to the coils 726, 728 and a second magnetic-field signal (e.g., “y” or noise cancellation signal) to a noise cancellation coil of a superconducting quantum interference device (SQUID) 206. The amplifier 202 is configured to lock without an external reference and may be a Perkins Elmer model 7265 DSP lock-in amplifier. This amplifier works in a “virtual mode,” where it locks to an initial reference frequency, and then removes the reference frequency to allow it to run freely and lock to “noise.”

A magnetic-field stimulus generator, such as an analog Gaussian white noise stimulus generator 200 is electrically coupled to the amplifier 202. The generator 200 is configured to generate a selected magnetic-field stimulus, e.g., analog Gaussian white noise stimulus at the coils 726, 728 via the amplifier 202. As an example, the generator 200 may be a model 1380 manufactured by General Radio.

An impedance transformer 204 is electrically coupled between the SQUID 206 and the amplifier 202. The impedance transformer 204 is configured to provide impedance matching between the SQUID 206 and amplifier 202.

The SQUID 206 is a low temperature direct element SQUID. As an example, the SQUID 206 may be a model LSQ/20 LTS dC SQUID available form Tristan Technologies, Inc (San Diego, Calif.) Alternatively, a high temperature or alternating current SQUID can be used. The coils 722, 724 (e.g., gradiometer) and the SQUID 206 (collectively referred to as the SQUID/gradiometer detector assembly) combined has a magnetic field measuring sensitivity of approximately 5 microTesla/√Hz. The induced voltage in the coils 722, 724 is detected and amplified by the SQUID 206. The output of the SQUID 206 is a voltage approximately in the range of 0.2-0.8 microvolts.

The output of the SQUID 206 is the input to a SQUID controller 208. The SQUID controller 208 is configured to control the operational state of the SQUID 206 and further condition the detected signal. As an example, the SQUID controller 208 may be an iMC-303 iMAG multi-channel SQUID controller manufactured by Tristan Technologies, Inc.

The output of the SQUID controller 208 is inputted to an amplifier 210. The amplifier 210 is configured to provide a gain in the range of 0-100 dB. A gain of approximately 20 dB is provided when noise cancellation node is turned on at the SQUID 206. A gain of approximately 50 dB is provided when the SQUID 206 is providing no noise cancellation.

The amplified signal is inputted to a recorder or storage device 212. The recorder 212 is configured to convert the analog amplified signal to a digital signal and store the digital signal. In one embodiment, the recorder 212 stores 8600 data points per Hz and can handle 2.46 Mbits/sec. As an example, the recorder 212 may be a Sony digital audiotape (DAT) recorder. Using a DAT recorder, the raw signals or data sets can be sent to a third party for display or specific processing as desired.

A lowpass filter 214 filters the digitized data set from the recorder 212. The lowpass filter 214 is an analog filter and may be a Butterworth filter. The cutoff frequency is at approximately 50 kHz.

A bandpass filter 216 next filters the filtered data sets. The bandpass filter 216 is configured to be a digital filter with a bandwidth between DC to 50 kHz. The bandpass filter 216 can be adjusted for different bandwidths.

The output of the bandpass filter 216 is the input to a Fourier transformer processor 218. The Fourier transform processor 218 is configured to convert the data set, which is in the time domain, to a data set in the frequency domain. The Fourier transform processor 218 performs a Fast Fourier Transform (FFT) type of transform.

The Fourier transformed data sets are the input to a correlation and comparison processor 220. The output of the recorder 212 is also an input to the processor 220. The processor 220 is configured to correlate the data set with previously recorded data sets, determine thresholds, and perform noise cancellation (when no noise cancellation is provided by the SQUID 206). The output of the processor 220 is a final data set representative of the spectrum of the sample's molecular low frequency electromagnetic emissions.

A user interface (UI) 222, such as a graphical user interface (GUI), may also be connected to at least the filter 216 and the processor 220 to specify signal processing parameters. The filter 216, processor 218, and the processor 220 can be implemented as hardware, software, or firmware. For example, the filter 216 and the processor 218 may be implemented in one or more semiconductor chips. The processor 220 may be software implemented in a computing device.

This amplifier works in a “virtual mode,” where it locks to an initial reference frequency, and then removes the reference frequency to allow it to run freely and lock to “noise.” The analog noise generator (which is produced by General Radio, a truly analog noise generator) requires 20 dB and 45-dB attenuation for the Helmholtz and noise cancellation coil, respectively.

The Helmholtz coil may have a sweet spot of about one cubic inch with a balance of 1/100^{th} of a percent. In an alternative embodiments, the Helmholtz coil may move both vertically, rotationally (about the vertical axis), and from parallel to spread apart in a pie shape. In one embodiment, the SQUID, gradiometer, and driving transformer (controller) have values of 1.8, 1.5 and 0.3 micro-Henrys, respectively. The Helmholtz coil may have a sensitivity of 0.5 Gauss per amp at the sweet spot.

Approximately 10 to 15 microvolts may be needed for a stochastic response. By injecting Gaussian white noise stimulus, the system has raised the sensitivity of the SQUID device. The SQUID device had a sensitivity of about 5 femtotesla without the noise. This system has been able to improve the sensitivity by 25 to 35 dB by injecting noise and using this stochastic resonance response, which amounts to nearly a 1,500% increase.

After receiving and recording signals from the system, a computer, such as a mainframe computer, supercomputer or high-performance computer does both pre and post processing, such by employing the Autosignal software product by Systat Software of Richmond Calif., for the pre-processing, while Flexpro software product does the post-processing. Flexpro is a data (statistical) analysis software supplied by Dewetron, Inc. The following equations or options may be used in the Autosignal and Flexpro products.

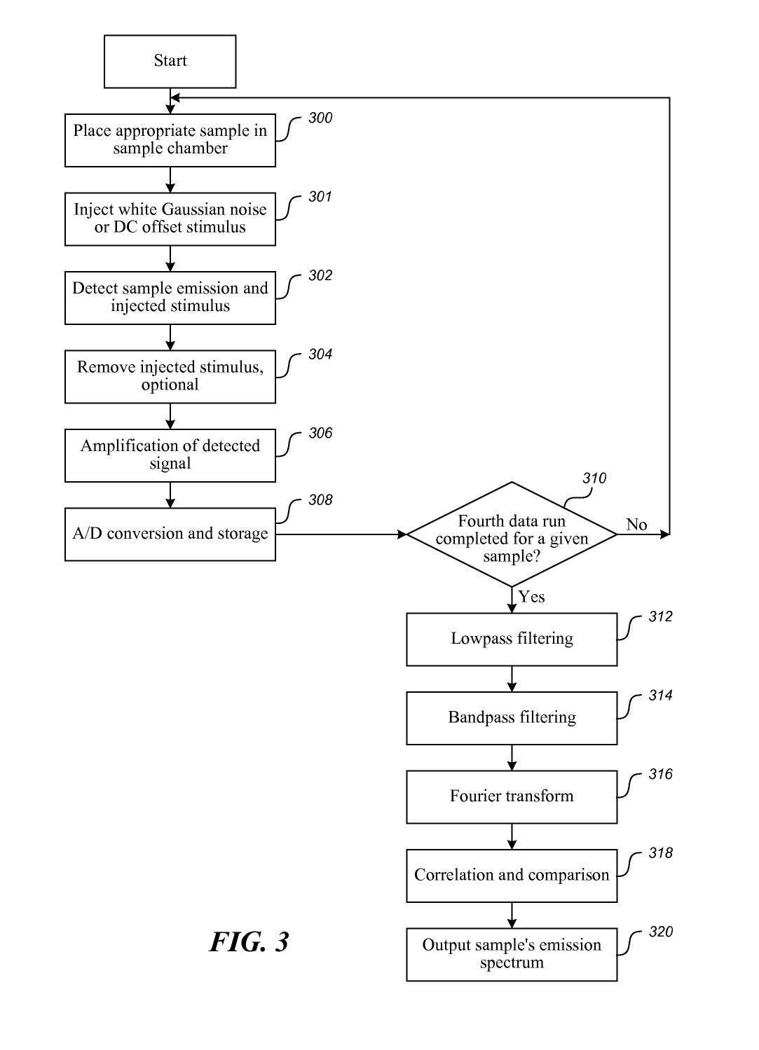

A flow diagram of the signal detection and processing performed by the apparatus is shown in FIG. 3. When a sample is of interest, typically at least four signal detections or data runs are performed: a first data run at a time t₁ without the sample, a second data run at a time t₂ with the sample, a third data run at a time t₃ with the sample, and a fourth data run at a time t₄ without the sample. Performing and collecting data sets from more than one data run increases accuracy of the final (e.g., correlated) data set. In the four data runs, the parameters and conditions of the system are held constant (e.g., temperature, amount of amplification, position of the coils, the Gaussian white noise and/or DC offset signal, etc.).

At block 300, the appropriate sample (or if it's a first or fourth data run, no sample), is placed in the apparatus, e.g., apparatus 700. A given sample, without injected Gaussian white noise or DC-offset stimulus, emits electromagnetic emissions in the DC-50 kHz range at an amplitude equal to or less than approximately 0.001 microTesla. To capture such low emissions, Gaussian white noise stimulus and/or DC offset is injected at block 301.

At block 302, the coils 722, 724 detect the induced voltage representative of the sample's emission and the injected magnetic stimulus. The induced voltage comprises a continuous stream of voltage values (amplitude and phase) as a function of time for the duration of a data run. A data run can be 2-20 minutes in length and hence, the data set corresponding to the data run comprises 2-20 minutes of voltage values as a function of time.

At block 304, the injected magnetic stimulus is cancelled as the induced voltage is being detected. This block is omitted when the noise cancellation feature of the SQUID 206 is turned off.

At block 306, the voltage values of the data set are amplified by 20-50 dB, depending on whether noise cancellation occurred at the block 304. And at—block 308, the amplified data set undergoes analog to digital (A/D) conversion and is stored in the recorder 212. A digitized data set can comprise millions of rows of data.

After the acquired data set is stored, at a block 310 a check is performed to see whether at least four data runs for the sample have occurred (e.g., have acquired at least four data sets). If four data sets for a given sample have been obtained, then lowpass filtering occurs at block 312. Otherwise, the next data run is initiated (return to the block 300).

After lowpass filtering (block 312) and bandpass filtering (at a block 314) the digitized data sets, the data sets are converted to the frequency domain at a Fourier transform block 316.

Next, at block 318, like data sets are correlated with each other at each data point. For example, the first data set corresponding to the first data run (e.g., a baseline or ambient noise data run) and the fourth data set corresponding to the fourth data run (e.g., another noise data run) are correlated to each other. If the amplitude value of the first data set at a given frequency is the same as the amplitude value of the fourth data set at that given frequency, then the correlation value or number for that given frequency would be 1.0. Alternatively, the range of correlation values may be set at between 0-100. Such correlation or comparison also occurs for the second and third data runs (e.g., the sample data runs). Because the acquired data sets are stored, they can be accessed at a later time as the remaining data runs are completed.

Predetermined threshold levels are applied to each correlated data set to eliminate statistically irrelevant correlation values. A variety of threshold values may be used, depending on the length of the data runs (the longer the data runs, greater the accuracy of the acquired data) and the likely similarity of the sample's actual emission spectrum to other types of samples. In addition to the threshold levels, the correlations are averaged. Use of thresholds and averaging correlation results in the injected Gaussian white noise stimulus component becoming very small in the resulting correlated data set.

Once the two sample data sets have been refined to a correlated sample data set and the two noise data sets have been refined to a correlated noise data set, the correlated noise data set is subtracted from the correlated sample data set. The resulting data set is the final data set (e.g., a data set representative of the emission spectrum of the sample) (block 320).

Since there can be 8600 data points per Hz and the final data set can have data points for a frequency range of DC-50 kHz, the final data set can comprise several hundred million rows of data. Each row of data can include the frequency, amplitude, phase, and a correlation value.

### III. Method of Identifying Optimal Time-Domain Signals for Transduction

The signals produced in accordance with the apparatus and methods described above may be further selected for optimal effector activity, when used to transduce an in vitro or mammalian system. As detailed in co-owned PCT application WO2008/063654 A3, sample-dependent signal features in a time-domain signal obtained for a given sample can be optimized by recording time-domain signals for the sample over a range of magnetic-field stimulus conditions, e.g., different voltage levels for Gaussian white noise stimulus amplitudes and/or DC offsets. The recorded signals are then processed to reveal signal features, and one or more time domain signals having an optimal signal-analysis score, as detailed below, are selected. The selection of optimized or near-optimized time-domain signals is useful because it has been found that transducing an in vitro or biological system with an optimized time-domain signal gives a stronger and more predictable response than with a non-optimized time-domain signal. That is, selecting an optimized (or near-optimized) time-domain signal is useful in achieving reliable, detectable sample effects when a target system is transduced by the sample signal.

In general, the range of injected white noise and DC offset voltages applied to the sample are such as to produce a calculated magnetic field at the sample container of between 0 to 1 G (Gauss), or alternatively, the injected noise stimulus is preferably between about 30 to 35 decibels above the molecular electromagnetic emissions sought to be detected, e.g., in the range 70-80-dbm. The number of samples that are recorded, that is, the number of noise-level intervals over which time-domain signals are recorded may vary from 10-100 or more, typically, and in any case, at sufficiently small intervals so that a good optimum signal can be identified. For example, the power gain of the noise generator level can be varied over 50 20 mV intervals.

Alternatively, stimulus signals other than Gaussian white noise and/or DC offset can be used for optimization of the recorded time-domain signal. Examples of such signals include scanning a range of sine wave frequencies, a square wave, time-series data containing defined non-linear structure, or the SQUID output itself. These signals may themselves be pulsed between off and on states to further modify the stimulus signal. The white noise naturally generated by the magnetic shields may also be used as the source of the stimulus signal.

Above-cited PCT application WO 2008/063654 describes five methods for scoring the time-domain signals produced as above: (A) a histogram bin method, (B) generating an FFT of autocorrelated signals, (C) averaging of FFTs, (D) use of a cross-correlation threshold, and (E) phase-space comparison. Of these, the most successful predictors of effective transduction signals have been the histogram bin method (A), and enhanced autocorrelation (EAC) method (B). The two preferred methods are discussed below.

### A. Histogram Method of Generating Spectral Information

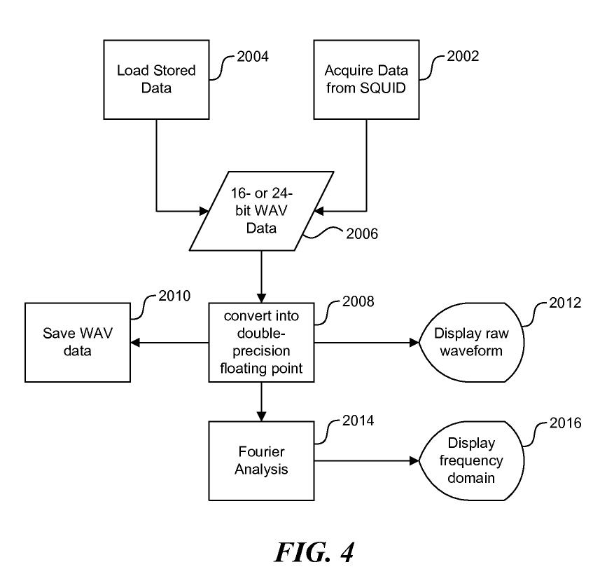

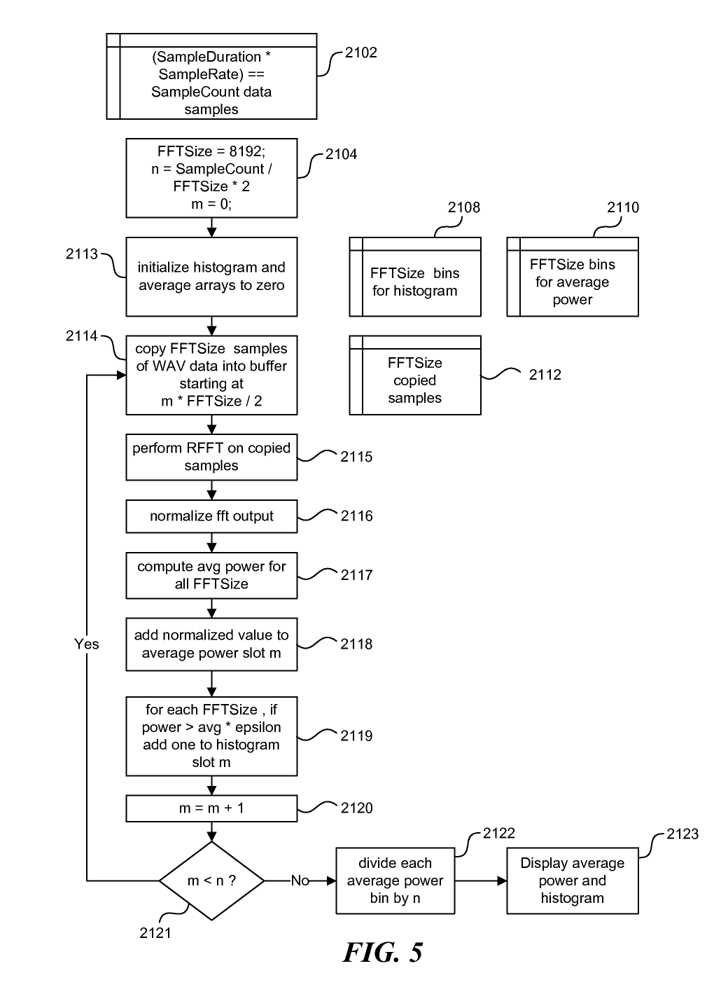

FIG. 4 is a high level data flow diagram in the histogram method for generating spectral information. Data acquired from the SQUID (box 2002) or stored data (box 2004) is saved as 16 or 24 bit WAV data (box 2006), and converted into double-precision floating point data (box 2008). The converted data may be saved (box 2010) or displayed as a raw waveform (box 2012). The converted data is then passed to the algorithm described below with respect to FIG. 5, and indicated by the box 2014 labeled Fourier Analysis. The histogram can be displayed at 2016.

FIG. 5 shows the general flow of the histogram scoring algorithm. The time-domain signals are acquired from an ADC (analog/digital converter) and stored in the buffer indicated at 2102. This sample is SampleDuration seconds long, and is sampled at SampleRate samples per second, thus providing SampleCount (SampleDuration*SampleRate) samples. The FrequencyRange that can be recovered from the signal is defined as half the SampleRate, as defined by Nyquist. Thus, if a time-series signal is sampled at 10,000 samples per second, the FrequencyRange will be 0 Hz to 5 kHz. One Fourier algorithm that may be used is a Radix 2 Real Fast Fourier Transform (RFFT), which has a selectable frequency domain resolution (FFTSize) of powers of two up to 2¹⁶. An FFTSize of 8192 is selected, to provide provides enough resolution to have at least one spectrum bin per Hertz as long as the FrequencyRange stays at or below 8 kHz. The SampleDuration should be long enough such that SampleCount>(2*) FFTSize*10 to ensure reliable results.

Since this FFT can only act on FFTSize samples at a time, the program must perform the FFT on the samples sequentially and average the results together to get the final spectrum. If one chooses to skip FFTSize samples for each FFT, a statistical error of 1/FFTSizê0.5 is introduced. If, however, one chooses to overlap the FFT input by half the FFTSize, this error is reduced to 1/(0.81*2*FFTSize)̂0.5. This reduces the error from 0.0110485435 to 0.0086805556. Additional information about errors and correlation analyses in general, consult Bendat & Piersol, “Engineering Applications of Correlation and Spectral Analysis”, 1993.

Prior to performing the FFT on a given window, a data tapering filter may be applied to avoid spectral leakage due to sampling aliasing. This filter can be chosen from among Rectangular (no filter), Hamming, Hanning, Bartlett, Blackman and Blackman/Harris, as examples.

In an exemplary method, and as shown in box 2104, we have chosen 8192 for the variable FFTSize, which will be the number of time-domain samples we operate on at a time, as well as the number of discrete frequencies output by the FFT. Note that FFTSize=8192 is the resolution, or number of bins in the range which is dictated by the sampling rate. The variable n, which dictates how many discrete RFFT's (Real FFT's) performed, is set by dividing the SampleCount by FFTSize*2, the number of FFT bins. In order for the algorithm to generate sensible results, this number n should be at least 10 to 20 (although other valves are possible), where more may be preferred to pick up weaker signals. This implies that for a given SampleRate and FFTSize, the SampleDuration must be long enough. A counter m, which counts from 0 to n, is initialized to zero, also as shown in box 2104.

The program first establishes three buffers: buffer 2108 for FFTSize histogram bins, that will accumulate counts at each bin frequency; buffer 2110 for average power at each bin frequency, and a buffer 2112 containing the FFTSize copied samples for each m.

The program initializes the histograms and arrays (box 2113) and copies FFTSize samples of the wave data into buffer 2112, at 2114, and performs an RFFT on the wave data (box 2115). The FFT is normalized so that the highest amplitude is 1 (box 2116) and the average power for all FFTSize bins is determined from the normalized signal (box 2117). For each bin frequency, the normalized value from the FFT at that frequency is added to each bin in buffer 2108 (box 2118).

In box 2119 the program then looks at the power at each bin frequency, relative to the average power calculated from above. If the power is within a certain factor epsilon (between 0 and 1) of the average power, then it is counted and the corresponding bin is incremented in the histogram buffer at 16. Otherwise it is discarded.

Note that the average power it is comparing to is for this FFT instance only. An enhanced, albeit slower algorithm might take two passes through the data and compute the average over all time before setting histogram levels. The comparison to epsilon helps to represent a power value that is significant enough for a frequency bin. Or in broader terms, the equation employing epsilon helps answer the question, “is there a signal at this frequency at this time?” If the answer is yes, it could due be one of two things: (1) stationary noise which is landing in this bin just this one time, or (2) a real low level periodic signal which will occur nearly every time. Thus, the histogram counts will weed out the noise hits, and enhance the low level signal hits. So, the averaging and epsilon factor allow one to select the smallest power level considered significant.

Counter m is incremented at box 2120, and the above process is repeated for each n set of WAV data until m is equal to n (box 2121). At each cycle, the average power for each bin is added to the associated bin at 2118, and each histogram bin is incremented by one when the power amplitude condition at 2114 is met.

When all n cycles of data have been considered, the average power in each bin is determined by dividing the total accumulated average power in each bin by n, the total number of cycles (box 2122) and the results displayed (box 2123). Except where structured noise exists, e.g., DC=0 or at multiples of 60 Hz, the average power in each bin will be some relatively low number.

The relevant settings in this method are noise stimulus gain and the value of epsilon. This value determines a power value that will be used to distinguish an event over average value. At a value of 1, no events will be detected, since power will never be greater than average power. As epsilon approaches zero, virtually every value will be placed in a bin. Between 0 and 1, and typically at a value that gives a number of bin counts between about 20-50% of total bin counts for structured noise, epsilon will have a maximum “spectral character,” meaning the stochastic resonance events will be most highly favored over pure noise.

Therefore, one can systematically increase the power gain on the magnetic-field stimulus input, e.g., in 50 mV increments between 0 and 1 V, and at each power setting, adjust epsilon until a histogram having well defined peaks is observed. Where, for example, the sample being processed represents a 20 second time interval, total processing time for each different power and epsilon will be about 25 seconds. When a well-defined signal is observed, either the power setting or epsilon or both can be refined until an optimal histogram, meaning one with the largest number of identifiable peaks, is produced.

Under this algorithm, numerous bins may be filled and associated histogram rendered for low frequencies due to the general occurrence of noise (such as environmental noise) at the low frequencies. Thus, the system may simply ignore bins below a given frequency (e.g., below 1 kHz), but still render sufficient bin values at higher frequencies to determine unique signal signatures between samples.

Alternatively, since a purpose of the epsilon variable is to accommodate different average power levels determined in each cycle, the program could itself automatically adjust epsilon using a predefined function relating average power level to an optimal value of epsilon.

Similarly, the program could compare peak heights at each power setting, and automatically adjust the noise stimulus power setting until optimal peak heights or character is observed in the histograms.

Although the value of epsilon may be a fixed value for all frequencies, it is also contemplated to employ a frequency-dependent value for epsilon, to adjust for the higher value average energies that may be observed at low frequencies, e.g., DC to 1,000. A frequency-dependent epsilon factor could be determined, for example, by averaging a large number of low-frequency FFT regions, and determining a value of epsilon that “adjusts” average values to values comparable to those observed at higher frequencies.

B. Enhanced autocorrelation (EAC)

In a second preferred method for determining signal-analysis scores, time-domain signals recorded at a selected noise stimulus are autocorrelated, and a fast Fourier transform (FFT) of the autocorrelated signal is used to generate a signal-analysis plot, that is, a plot of the signal in the frequency domain. The FFTs are then used to score the number of spectral signals above an average noise level over a selected frequency range, e.g., DC to 1 kHz or DC to 8 kHz.

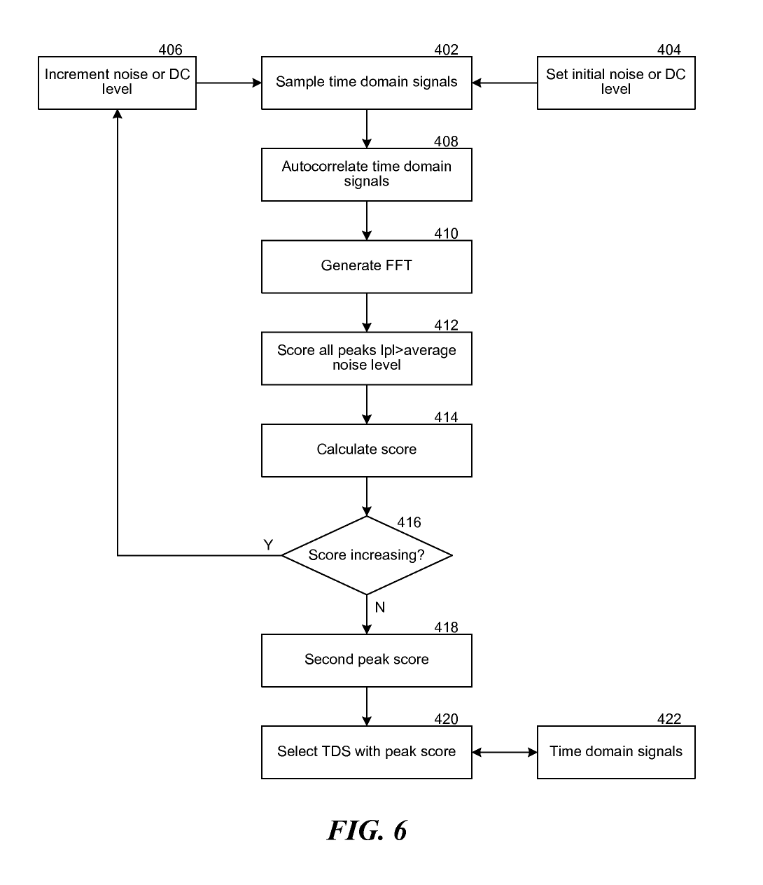

FIG. 6 is a flow diagram of steps carried out in scoring recorded time-domain signals according to this second embodiment. Time-domain signals are sampled, digitized, and filtered as above (box 402), with the gain on the magnetic-field stimulus level set to an initial level, as at 404. A typical time domain signal for a sample compound 402 is autocorrelated, at 408, using a standard autocorrelation algorithm, and the FFT of the autocorrelated function is generated, at 410, using a standard FFT algorithm.

An FFT plot is scored, at 412, by counting the number of spectral peaks that are statistically greater than the average noise observed in the autocorrelated FFT and the score is calculated at 414. This process is repeated, through steps 416 and 406, until a peak score is recorded, that is, until the score for a given signal begins to decline with increasing magnetic stimulus gain. The peak score is recorded, at 418, and the program or user selects, from the file of time-domain signals at 422, the signal corresponding to the peak score (box 420).

As above, this embodiment may be carried out in a manual mode, where the user manually adjusts the magnetic stimulus setting in increments, analyzes (counts peaks) from the FFT spectral plots by hand, and uses the peak score to identify one or more optimal time-domain signals. Alternatively, one or more aspects of the steps can be automated.

### IV. Transduction Apparatus and Protocols

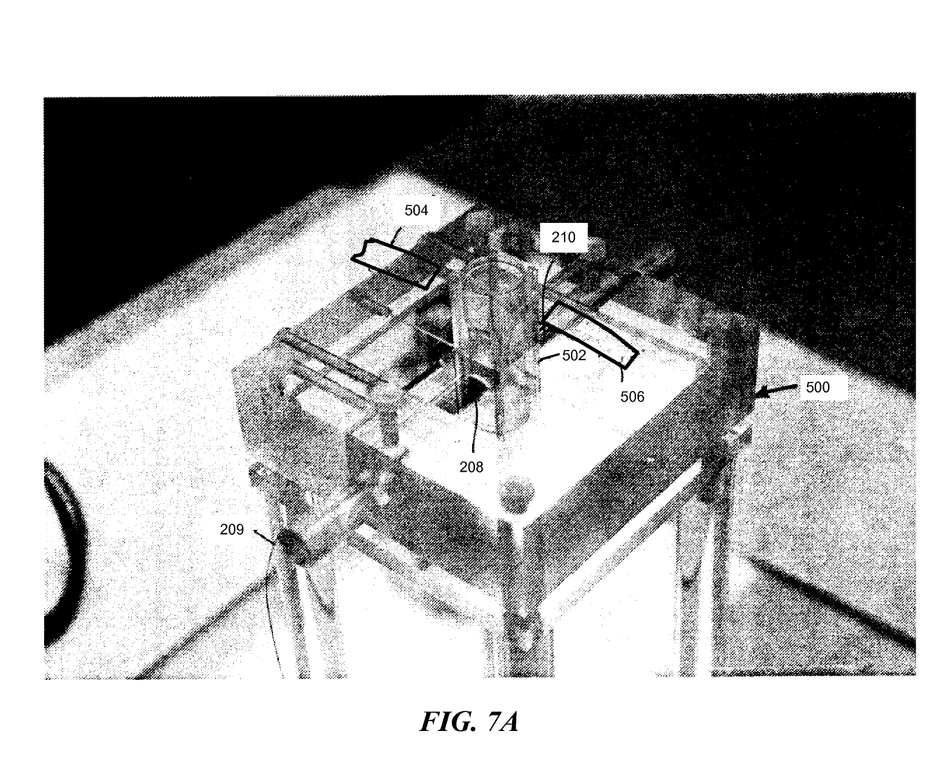

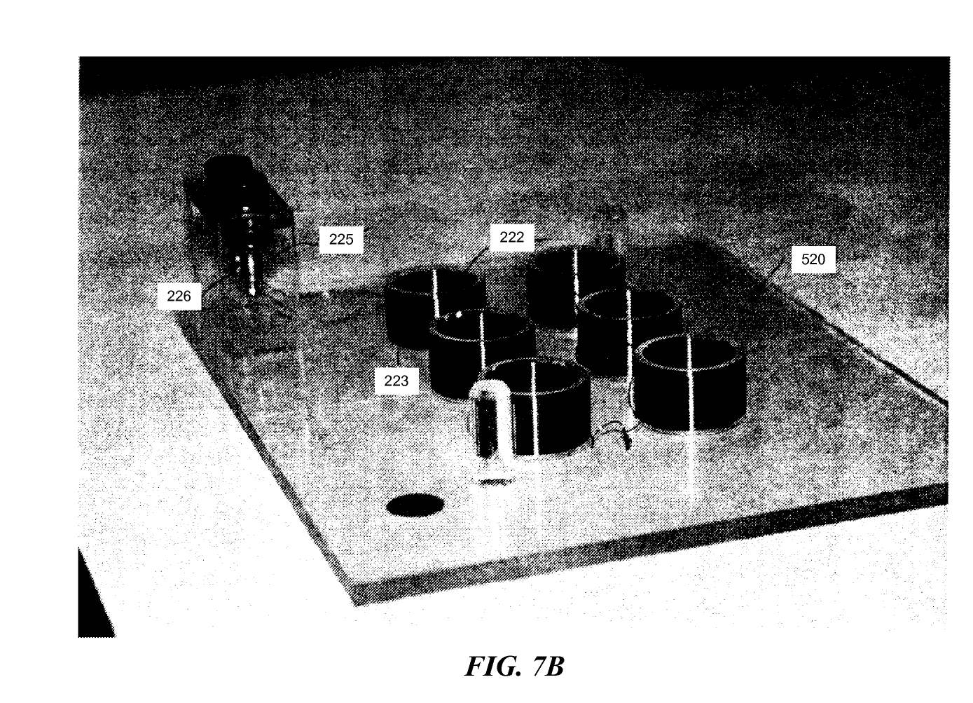

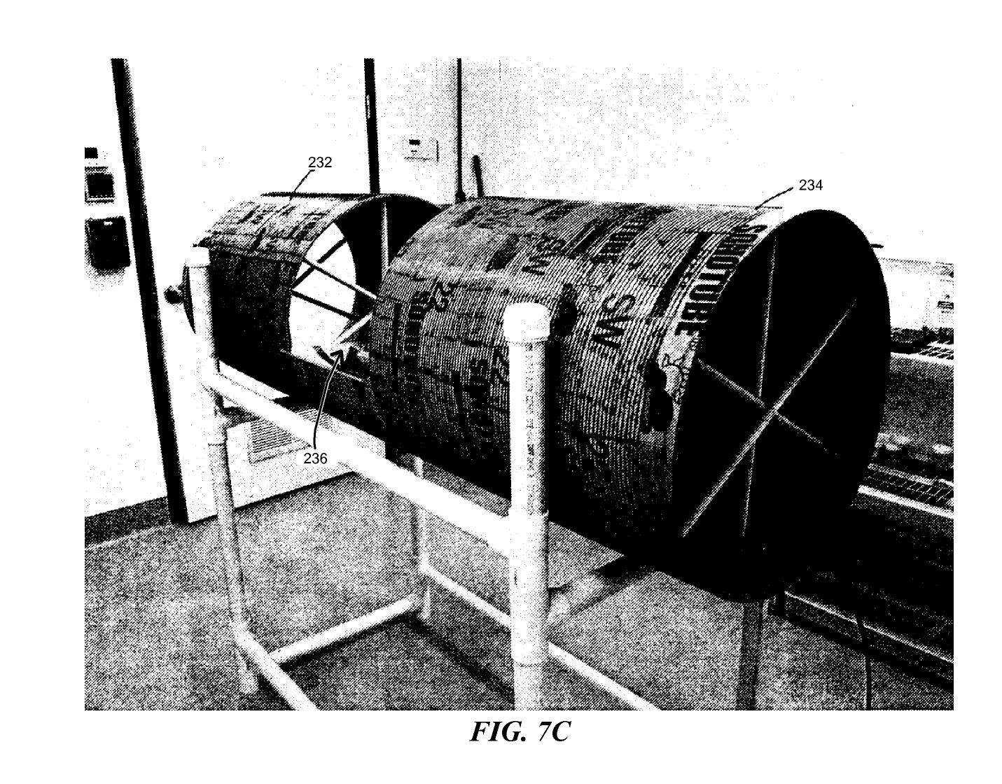

This section describes equipment and methodology for transducing a sample with signals generated and selected according to the methods described in Sections I and II above. The signals are used in one of the transducers described below to produce a compound-specific response in various in vitro or mammalian systems. [0147] One general type of transducer, shown at 500 in FIG. 7A, is designed for detecting changes in an optical characteristic of the system in response to transduction. This transducer includes an optically transparent cell 502, which serves as the transduction station in the transducer, and optical detection components, including a light source 504 and a photodetector 506, for measuring light-induced changes in a sample target produced during transduction. This type of transducer is employed, for example, in the studies reported below on the effect of taxol time-domain signals on tubulin polymerization, as monitored by a change in light scattering within the cell. One exemplary sample cell is a 70 μL volume quartz cuvette. Transduction coils 208, 210 located at opposite end regions of the cell were engineered and manufactured by American Magnetics to provide uniform magnetic field strength between coils, and leads for the two coils are shown at 209, 211. Each coil consists of 50 turns of #39 gauge (awg) square copper magnet wire, enamel coated, with about a diameter 7.82 mm air core.

A transducer suitable for use in transducing cells in a plurality (in this case, six) of culture or plated samples is shown at 520 in FIG. 7B. In this embodiment, the transduction station is occupied by one culture plates, such as plates 222. The plates are typically conventional plastic or glass Petri dishes of 9.4 cm², where each plate is surrounded by a separate transduction coil, such as coil 223, and all of the coils are connected separately by leads 225, 226. Similar to the first embodiment, the transduction coils were engineered and manufactured by American Magnetics to provide uniform magnetic field strength within each well. Each coil consists of 49 turns of #49 gauge (awg) square copper magnet wire, enamel coated, with about a diameter 26 mm air core.

A third transducer, illustrated at 230 in FIG. 7C, is designed for applying transducing signals to one or more laboratory animals, such as mice, for studying the effects of a transduction signal on the animals. The transducer includes a pair of coils 232, 234, positioned at opposite end regions of a non-metallic cage, which serves as a transduction station 236. As above, the coils were engineered and manufactured by American Magnetics to provide uniform magnetic filed strength through the interior of the cage. Each coil consists of 94 turns of #22 gauge (awg) square copper magnet wire, enamel coated, with about a diameter 22 in air core.

In another general embodiment of a transducer equipment, several Helmholtz coil pairs may be constructed to be orthogonal to one another. This configuration would allow considerable flexibility in controlling the structure of the magnetic field applied to a sample. For example, a static magnetic field could be applied along one axis, and a varying magnetic field applied along another axis. The transducers described above are placed in a shielded enclosure for the purpose of minimizing uncontrolled extraneous fields from the environment in the region where the sample is placed. [0151] In one embodiment of the shielding, the transduction equipment is located within a much larger enclosure, a least 3 times larger than the transduction equipment. This large container is lined with copper mesh attached to Earth ground. Such a container is commonly called a “Faraday cage”. The copper mesh attenuates external environmental electromagnetic signals that are greater than approximately 10 kHz.

In a second embodiment of the shielding, the transduction equipment is located within a large enclosure constructed of sheet aluminum or other solid conductor with minimal structural discontinuities. Such a container attenuates external environmental electromagnetic signals that are greater than approximately 1 kHz.

In a third embodiment of the shielding, the transduction equipment is located within a very large set of three orthogonal Helmholtz coil pairs, at least 5 times larger than the transduction equipment. A fluxgate magnetic sensor container is located near the geometric center of the Helmholtz coil pairs, and somewhat distant from the transduction equipment. Signal from the fluxgate sensor is input to a feedback device, such as a Lindgren, Inc. Magnetic Compensation System, and a feedback current used to drive the Helmholtz coils, forcing a region within the Helmholtz coils to be driven to zero field. Since the Helmholtz coil pairs are very large, this region is also correspondingly large. Furthermore, since the transduction equipment uses relatively small coils, their field does not extend outward sufficiently to interfere with the fluxgate sensor. Such a set of Helmholtz coil pairs attenuates external environmental electromagnetic signals between 0.001 Hz and 1 kHz.

In a fourth embodiment of the shielding, the transduction equipment may be located in either a copper mesh or aluminum enclosure as mentioned above, and that enclosure itself located within the set of Helmholtz coil pairs mentioned above. Such a configuration can attenuate external environmental electromagnetic signals over their combined ranges.

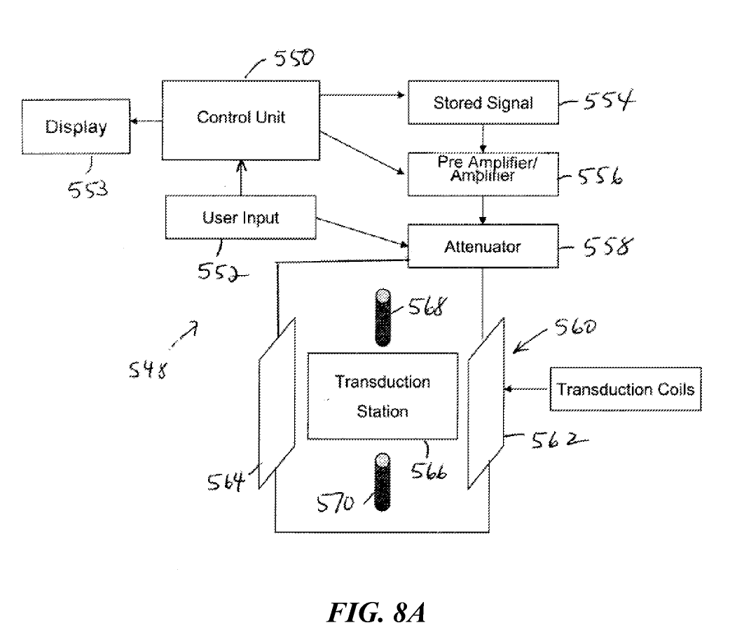

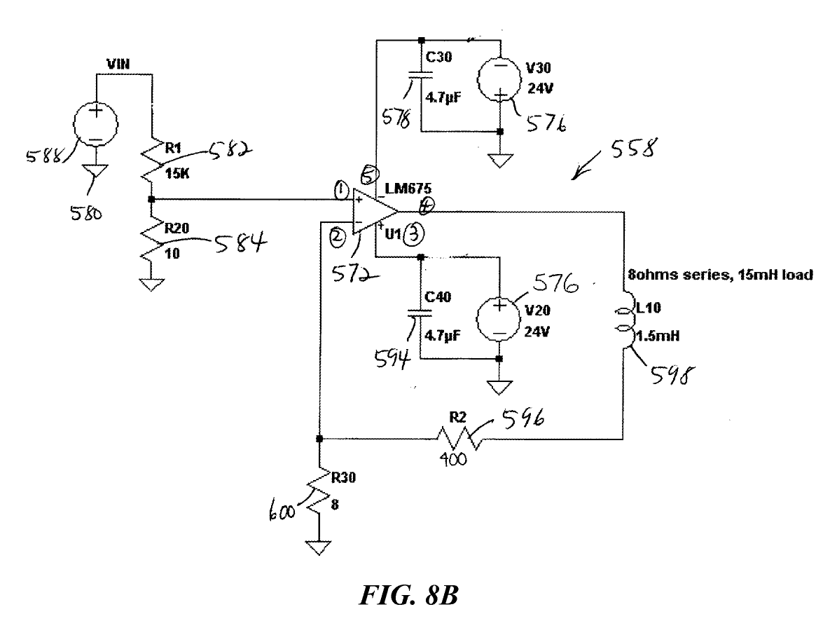

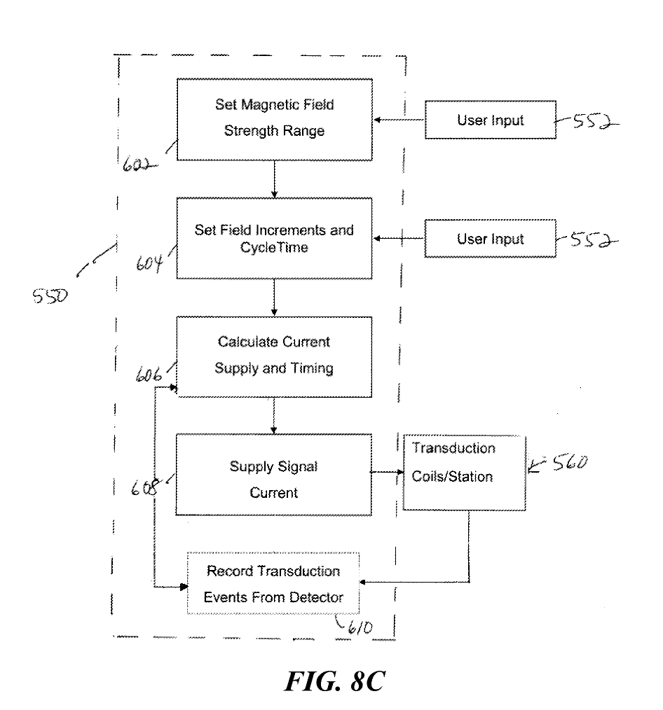

Each of the transducers described above forms part of a transduction system that includes components for converting a time-domain signal to a signal-related magnetic field at the transduction station in the transducer. FIG. 8A illustrates a general transduction system 548 having a transducer 560 composed of a pair of transduction coils 562, 564 on opposite ends of a transduction station 566. The transducer shown in the figure also includes photodetector components 568, 570, although these components are only needed where an optical transduction event is being measured.

A control unit 550 in the system is designed to receive user input from an input device 552, and display input information and system status and to the user at a display 553. As will be considered in FIG. 8C below, the user input typically includes information specifying the magnetic field strength or range or magnetic field strengths that will be applied during transduction operation information, specifying various timing variables, such as field-increment and field-cycle times, as well as total transduction time, as will be considered below. Based on this input, the control unit calculates settings that will be applied to the signal-amplifying and attenuating components in the system to achieve the desired transduction magnetic field strengths over the selected time periods.

A source of stored time-domain signal in the system is indicated at 554. Where the time-domain signal is recorded on a CD, the signal source includes the CD and a CD player, and as seen, is activated by the control unit. The signal source is connected to a conventional pre-amplifier/amplifier 556 also under the control of unit 552, which outputs an amplified signal voltage to an attenuator 558, also under the control of unit 550. As will be seen below with reference to FIG. 8B, the purpose of the attenuator is to convert signal voltage output from amplifier 556 to a signal current output, and to attenuate the output current to the transducer coils to produce a selected range of magnetic field strengths or a selected magnetic field. According to one embodiment of the invention, the attenuator can be set to produce selected magnetic fields having very low field strengths, in the range 10⁻⁸ to 10⁻¹¹ Tesla, although the range of producible field strengths may be much greater, e.g., 10⁻⁴ to 10⁻¹¹ Tesla.

In one general embodiment, the system is set by the user to supply voltage and current settings to the amplifier, preamplifier and attenuator to achieve incremental magnetic fields from about 10⁻⁴ to 10⁻¹¹ Tesla, over about 50 increments, where the settings for each increment are maintained for 1-5 seconds and the system continuously cycles through the range of field strengths over a user-selected transduction period, e.g., 20 minutes up to several days.

The signal is supplied to the electromagnetic coils 562 and 564 through separate channels, as shown. In one embodiment, a Sony Model CDP CE375 CD Player is used. Channel 1 of the Player is connected to CD input 1 of Adcom Pre Amplifier Model GFP 750. Channel 2 is connected to CD input 2 of Adcom Pre Amplifier Model GFP 750. CD's are recorded to play identical signals from each channel. Alternatively, CD's may be recorded to play different signals from each channel. A Gaussian white noise source can be substituted for signal source 554 for use as a white-noise transduction control. Although not shown here the system may include various probes for monitoring conditions, e.g., temperature within the transduction station.

The circuit diagram for an embodiment of attenuator 558 in FIG. 8A is shown in FIG. 8B, including a power amplifier 572 such as the National Semiconductor LM675 Power Operational Amplifier. The power amplifier 572 provides wide bandwidth and low input offset voltage, and is suitable for DC or AC applications, among other benefits. The power amplifier 572 is connected via pin 1 to an input Voltage 588, which is connected to ground 580 either directly or via one or more resistors (582, 584) acting to divide the input. Pin 2 is connected to ground, via a resistor 600. A DC power source 576, such as a regulated and filtered 24 Volt DC power source in parallel with capacitors 578 and 594, is connected to the power amplifier 572 at pin 3 and pin 5. The output of the amplifier (pin 4) is connected to an inductor 598, such as an 8.5 Ohm inductor.

Typical attenuation for such a circuit is approximately 90 dB. However, connecting the inductor 150 to ground 600 via a small resistance, such as the 400 Ohm resistor 596, provides additional attenuation, enabling the system to produce low output currents, as well as other benefits. The system may vary the attenuation by varying the value of the resistor 596, which in turn varies the output current. Additionally, the system may implement a low pass RC filter in series between the inductor 598 and ground 600 to eliminate or minimize self oscillation caused by any self generated tones within the circuit.

More generally, transduction by an incremented magnetic field produced by a signal current rather than signal voltage, and/or calculated to produce a selected range within 10⁻⁴ to 10⁻¹¹ Tesla, e.g., 10⁻⁶ to 10⁻¹¹ Tesla, and 10⁻⁸ to 10⁻¹¹ Tesla, represents an improved transduction method over earlier methods employing magnetic fields generated by signal voltage and/or at constant magnetic filed strength and/or at field strengths greater than about 10⁻⁸ Tesla.

The operational features of the transduction system 548 in FIG. 8A are illustrated in FIG. 8C, where the control unit, signal source, pre-amp and amp, and attenuator are indicated by the dashed line box 550. The transduction system 560 in the figure may be, for example, any of the three coil configurations shown in FIGS. 7A-7C, or variants thereof. As seen, the control unit is initially set by user input at 552 to a specified magnetic-field strength or incremented field-strength range desired at the transductions coils (box 602), and also set by the user to desired field increments and cycle times (box 604). For example, the user may specify a constant magnetic field strength, typically between 10⁻⁴ to 10⁻¹¹ Tesla, e.g., 10⁻⁸, 10⁻⁹, 10⁻¹⁰, or 10⁻¹¹ Tesla, or an incremented range of magnetic field strengths between 10⁻¹ to 10⁻¹¹ Tesla, such as a range between 10⁻⁴ to 10⁻¹¹ Tesla or between 10⁻⁸ to 10⁻¹¹ Tesla, e.g., 10⁻⁸ to 10⁻¹¹ Tesla. For a constant field strength, the user may then input desired “on” and “off” periods and total transduction period, for example, 5 minutes “on” 1 minute “off” over a total transduction period of 1 24 hours. Where an incrementing field-strength range is initially selected, the user will additionally specify the field-strength increments and total increment times, for example, 50 equal increments over 10⁻⁴ to 10⁻¹¹ Tesla, at increment times of 12 second each, for a total cycle time of ten minutes. In the incremented field strength operation, the control unit preferably operates to place a short “off” interval, e.g., one millisecond, between each incremented “on” interval, so that the target is exposed to discrete pulses of incremented magnetic pulses within each cycle. One preferred transduction coil configuration is composed of two. The magnetic field strength within the coil environment, as a function of the current level of the applied time-domain signal, can be calculated by well-known methods, for example, as indicated at box 606 in the figures, and as detailed on pages 122 to 142 of Applications of Maxwell's Equations, Cochran, J. F. and Heinrich, B., December, 2004. This calculation is done at 606 in the control unit. In one preferred embodiment, the signal current applied to the coils is incremented every 1-5 seconds, in 0.5 to 99.5 dB increments of magnetic field strength, to produce a calculated magnetic field strength that begins at nominal 10⁻¹¹ Tesla, and over a range of 1 to 99 steps, achieves a nominal maximum field strength of 10⁻⁸ Tesla, at which point a new cycle of magnetic-field pulses over the same range is begun. The interval between successive equal intensity magnetic-field pulses is preferably in the range of 1-100 sec.

The transduction parameters, i.e., the selected transduction conditions to which the system is exposed are (i) the current of the applied time-domain signal, (ii) the duration of applied signal, and (iii) the scheduling of the applied signal. The applied current may be over a range from slightly greater than zero to up to about 1000 mAmps. The total time of transduction may be from a few minutes to up to several days.

The box indicated at 608 in FIG. 8C includes the signal source, pre-amp and amp, and attenuator shown in FIG. 8A. These components are activated and controlled by the control unit to supply the desired current, current increments, cycle and total transduction times stored in the unit. The current output from the attenuator is delivered to the transduction coil(s) 560, as indicated, to produce the desired magnetic-filed strength in the transduction station. Where the course of transduction events can be monitored by a change in the optical (or other measurable) change in the target system, this information is fed to a component 610 in the control unit, and this information may be used to control transduction conditions, by feedback to component 606, and/or displayed to the user for purposes of manually controlling transduction conditions.

As will be seen below, and in accordance with one aspect of the invention, optimal effector time-domain signals, and optimized transduction conditions for transducing a mammalian system can be identified by transduction studies with a simplified in vitro analog of the mammalian system.

### V. Time-Domain Signals Having a Defined Mechanism of Action

As detailed in the above-identified '654 PCT application, the time-domain signals identified by the scoring algorithm above may be further selected for effectiveness in a mammalian system by testing each of the high-scoring signals in an in vitro system designed to serve as a simplified model that mirrors the interaction of the agent with a biochemical target in a more complex mammalian system. More specifically, the time-domain signal is selected for its ability to affect a target in vitro system through a mechanism of action by which a defined compound is known or presumed to have a desired therapeutic effect in an in vivo animal system. This section considers two such time-domain signals generated and selected for a specific mechanism of action that has important therapeutic application.

A first embodiment is a time-domain signal effective, when applied to a transduction coil in the system described above, is effective stabilize microtubule formation in a tubulin suspension, mimicking the mechanism of action of the class of taxane-like drugs. The studies reported below illustrate a time-domain signal capable of stabilizing microtubule polymerization, both in a tubulin suspension and in a cell culture system, similar to the effect of a taxane compound (taxol). As will be seen in Section V below, the signal is also effective in inhibiting tumor growth in tumor-bearing mice, again similar to an effect seen taxol administration.

A second embodiment is a time-domain signal effective, when applied to target cultured cells, of inhibiting mRNA expression, and corresponding protein expression, through an siRNA inhibitory mechanism that may involve production of an intermediary RNAi a compound.

### A. Time-Domain Signal Having a Tubulin-Stabilization Mechanism of Action

In one group of in vitro tests, time-domain signals were obtained by recording low-frequency signals from a sample of taxol suspended in Cremophore™ 529 ml and anhydrous ethanol 69.74 ml to a final concentration of 6 mg/ml. The signals were recorded with injected DC offset, at noise level settings between 10 and 241 mV and in increments of 1 mV. A total of 241 time-domain signals over this injected-noise level range were obtained, and these were analyzed by an enhanced autocorrelation algorithm detailed above, yielding 8 time-domain taxol signals for further in vitro testing. One of these, designated signal M2(3) was among the most effective of the 8 signals in the in vitro transduction studies described below.

The M2(3) and a number of other high-score signals were tested for their ability to stabilize tubulin polymerization, employing the transduction system described above with reference to FIG. 7A. The in vitro test for selecting the most effective time-domain signal that was chosen was a standard tubulin aggregation assay used for determining the tubulin assembly activity of an added compound. This assay has been described, for example, in Shelanski, M. L., Gaskin, F. and Cantor, C. R. (1973). Microtubule assembly in the absence of added nucleotides. Proc. Natl. Acad. Sci. U.S.A. 70, 765-768; and Lee, J. C. and Timasheff, S. N. (1977). In vitro reconstitution of calf brain microtubules: effects of solution variable. Biochemistry, 16, 1754-1762.

The M2(3) and a number of other high-score taxol signals were tested for their ability to stabilize tubulin polymerization. The tubulin-assembly reaction was carried out by exposing the tubulin in GPEM buffer, at a concentration of 1.5 mg/ml to the following polymerization conditions: (i) a buffer (control), (ii) tubulin alone (2nd control); (iii) taxol, added to a final concentration of 4 μM, and (iv) the M2(3) time-domain signal from above, applied to the transduction coils over a 10 minute period at an incremented transduction current calculated to produce an incremented magnetic field strength that cycles between 10⁻⁴ and 10⁻¹¹ Tesla, over 60 equal increments (approximately 10 mAmp change/increment), where each increment was pulsed for 1 second, followed by a brief cessation, giving a total cycle time of about 1 minute, and these cycles were repeated for the duration of the study, e.g., 10 minutes. Change in optical absorption at 340 nm was measured continuously for each sample, and the OD340 data was used to calculate a rate of tubulin polymerization (dA/minute at 340 nm) at each one minute interval during the transduction study.

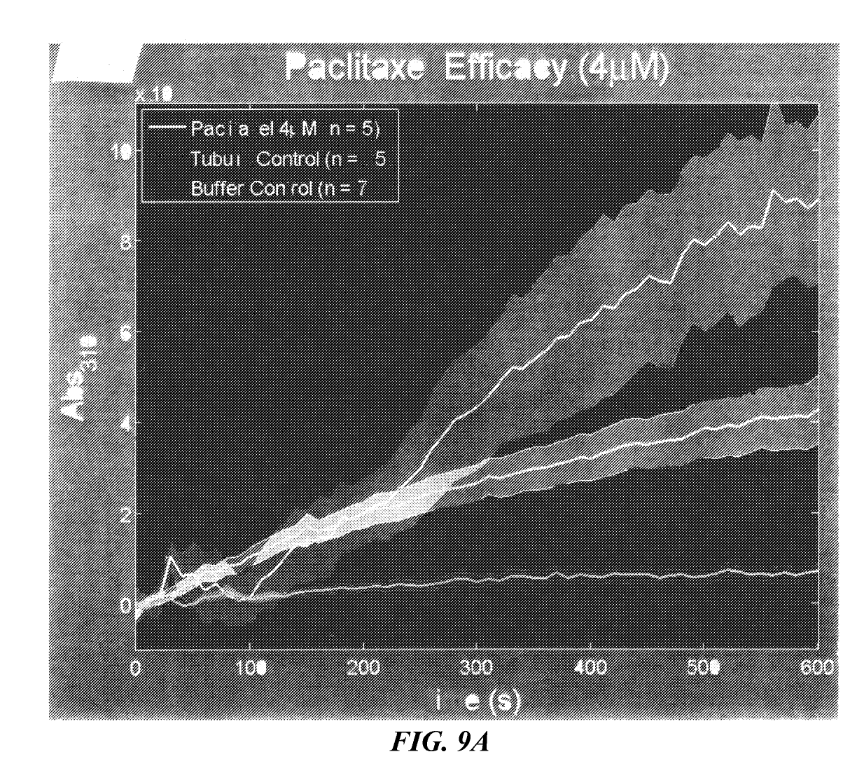

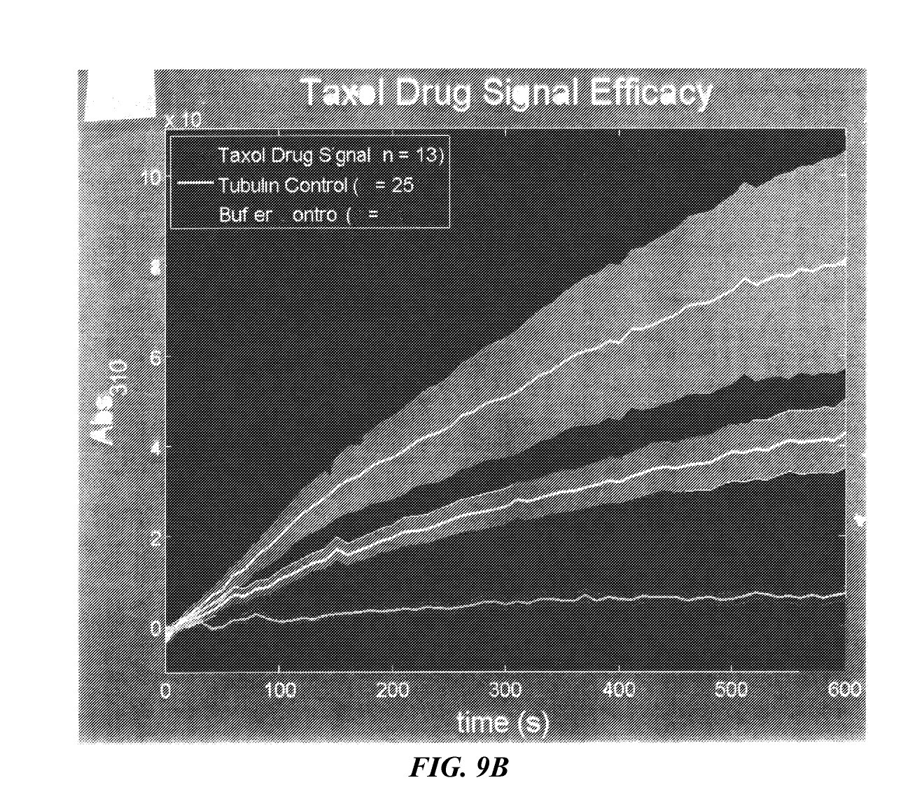

The results of the study, expressed as rate or tubulin polymerization over the first ten minutes of the study, are shown in FIGS. 9A and 9B. The lower trace envelop in the two figures shows the variation in absorbance for 7 separate samples for the buffer control. The middle trace in the two figures shows the change in absorbance for 25 tubulin control samples, i.e., tubulin in the absence of either taxol of taxol signal. The upper trace in FIG. 9A shows the increase in absorbance, evidencing tubulin polymerization for 5, for tubluin sample containing 4 μM taxol. As seen, there was an initial rise and fall in tubulin formation in the first minute after addition of taxol believed to be related to a “pre-polymerization” event characteristic of taxol-induced tubulin polymerization. Overall, absorbance increased more than twofold over the tubulin control.

The upper trace in FIG. 9B shows the increase in absorbance observed when tubulin samples (a total of 13) were transduced by the taxol-derived M2(3) time-domain signal, in the absence of any added taxol. As with the taxol effect seen in FIG. 9A, the signal transduction with the taxol-derived signal produced an approximately twofold increase in polymerization compared with the tubulin control over the ten-minute transduction period. Interestingly, and unlike the taxol-induced effect, a steady the increase in tubulin polymerization was seen from throughout the study.

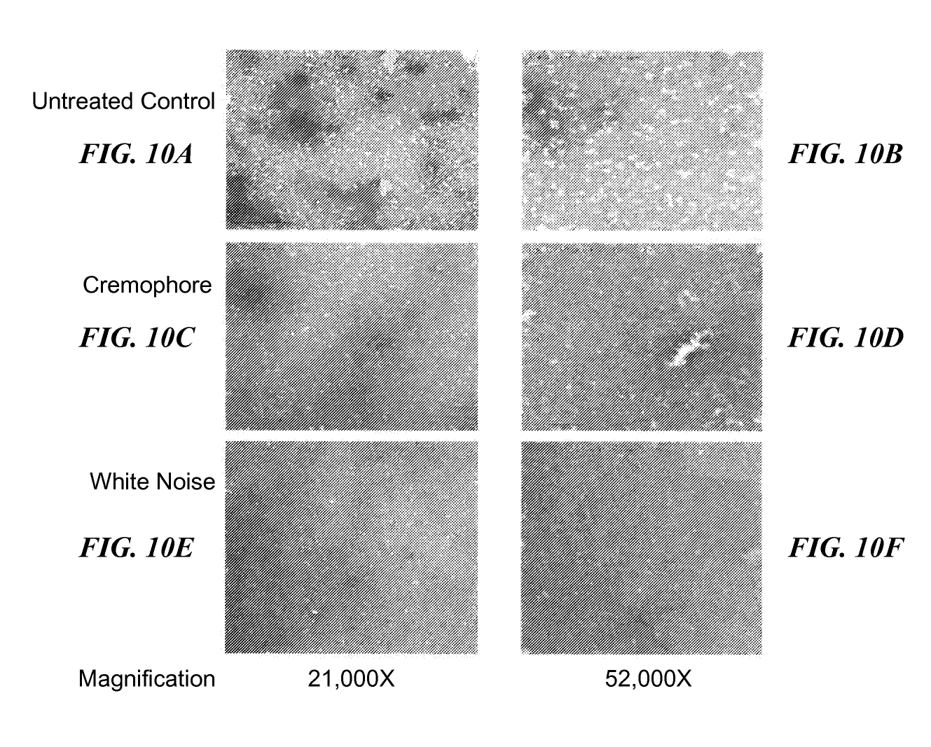

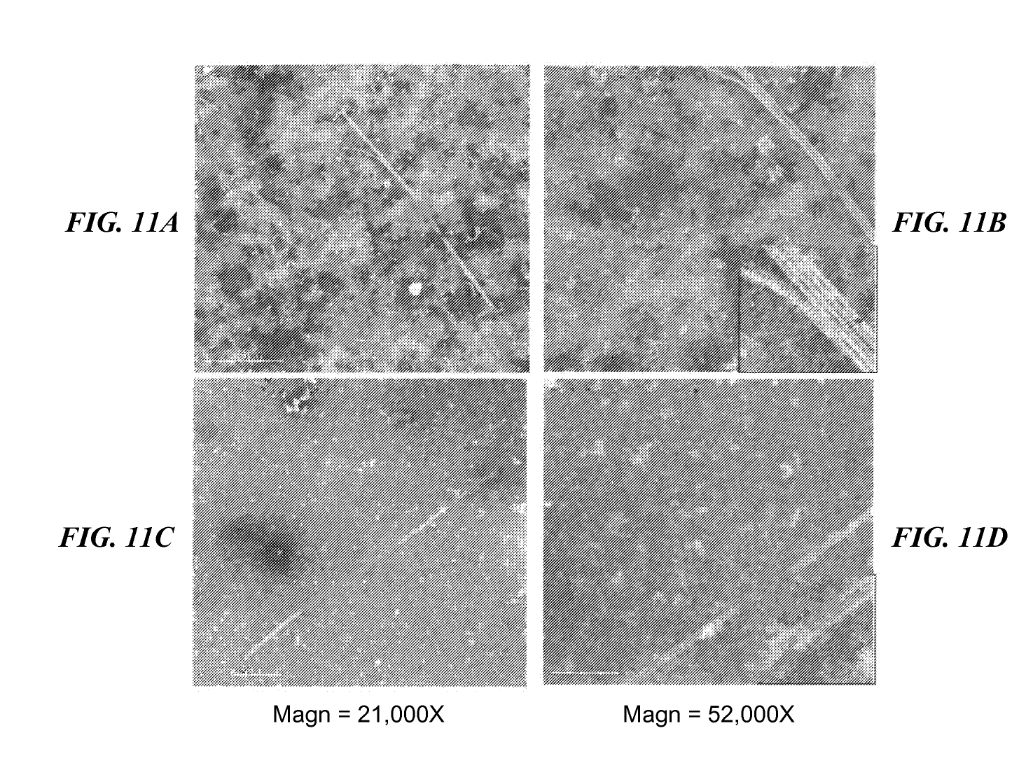

The assay samples from above were examined by electron microscopy to confirm that the nature of the microtubule polymers formed in the assay, either by the addition of taxol or the taxol-derived time-domain signal. FIGS. 10A-10F are photo electron micrographs, taken at 21,000× and 52,000× magnification, of a tubulin suspension for untreated control (FIGS. 10A and 10B), cremaphore alone, FIGS. 10C, 10D), and the tubulin suspension exposed to the magnetic field of a white-noise signal. Each sample was allowed to incubate in the system cell for 10 minutes under the conditions described, then placed on ice and immediately after cooling, placed on an EM grid and fixed for with stain. No evidence of significant tubulin formation is observed in any of these samples.

FIGS. 12A-12D show the morphology of the tubulin assay samples after 10 minutes exposure to 4 μM taxol, where the samples were cooled after 10 minutes and prepared for EM as above. A number of well-developed microtubular structures, including multi-lamellar structures are seen.

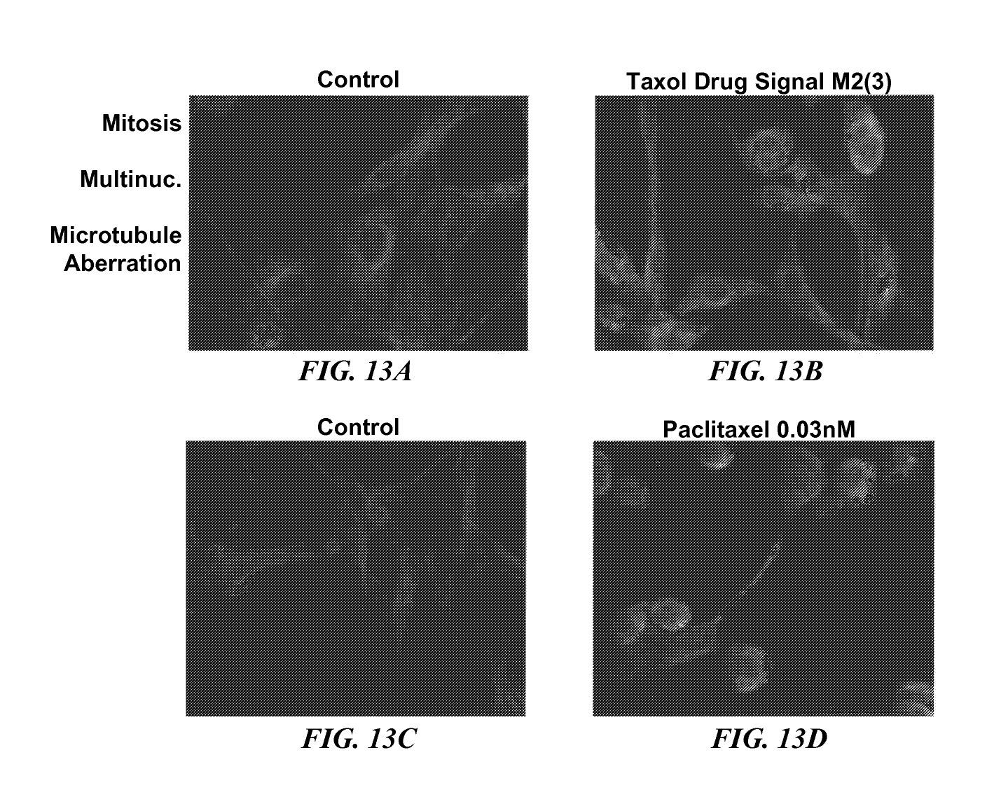

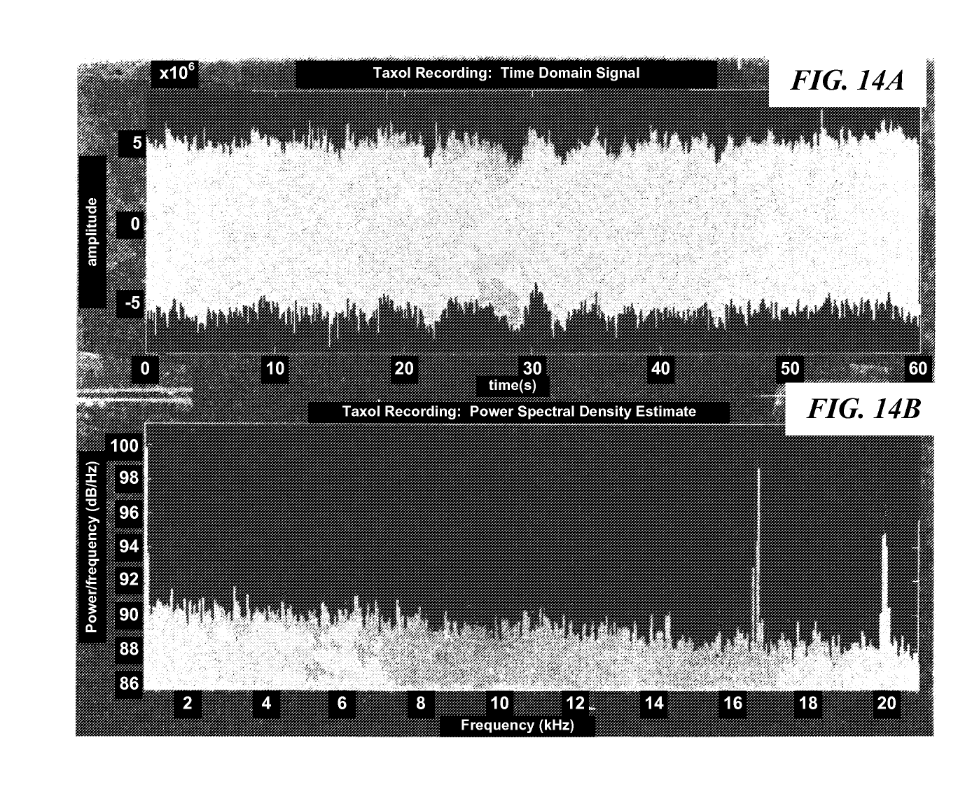

FIGS. 13A-13D show the morphology of the tubulin assay samples after 10 minutes exposure to the above M2(3) taxol-derived time-domain signal. 4 μM taxol, where the samples were cooled after 10 minutes and prepared for EM as above. As with the sample exposed to taxol itself, the samples here show a number of well-developed microtubular structures, including multi-lamellar structures.

To investigate whether the similar mechanism of action seen for taxol and a taxol-derived time-domain signals in a tubulin suspension would also be observed in cultured cells, U-87 glioblastoma cells were plated in DMEM medium, and allowed to grow to a density of 4×10^{X} cells/ml under standard culture conditions at 37° C. The culture plates were then divided into three groups of ______ plates each. A control group was incubated under the same conditions for an additional 72 hours, then stained by a standard DAPI nuclear stain and anti-tubulin antibody with secondary antibody tagged with Alexa488 for viewing by fluorescence microscopy. Taxol was added to the plates in the taxol-compound every 24 hours over a 72 hour incubation period, to a final taxol concentration of 0.03 nM taxol, and incubated under the same conditions, then stained and viewed as above. The 1 plate in a signal-transduced group were placed in the cell-plate transduction system described above in FIG. 7B, and transduced with the M2(3) signal for the same 72 hour period, using incremented transduction field strengths and cycle time employed in the tubulin assay.

The cells of the three groups were examined for microtubule cytoskeleton aberrations, mitosis and multinucleation. FIGS. 13A and 13C show control cells (no taxol or signal exposure). FIG. 13B shows cells exposed to the M2(3) signal. Multinucleation and other nuclear abnormalities are seen in several of the cells. Similar nuclear abnormalities are seen in the cells treated with taxol compound. The results strongly indicate that the mechanism of action of the M2(3) signal in stabilizing microtubule polymerization is at work in actively dividing cells as well.

A 60-second portion of the M2(3) time domain signal used in the studies above is shown in FIG. 14A, and the signal's power spectral density, generated by the enhanced autocorrelation method detailed in Section IIIB above, is seen in FIG. 14B. The latter figure illustrates spectral features associated with the time-domain signal, and also how the signal may be scored, by measuring the number and amplitude of peaks above a given threshold power/frequency level.

As discussed above, the signal is stored on a compact disc or any other suitable storage media for analog or digital signals and supplied to the transduction system during a signal transduction operation, for effecting a microtubule stabilization response in the target system. The signal carried on the compact disc is representative, more generally, of a tangible data storage medium having stored thereon, a low-frequency time domain signal effective to produce a magnetic field capable of stabilizing microtubule formation in an in vitro tubulin assay containing a suspension of tubulin, when the signal is supplied to electromagnetic transduction coil(s) at a signal current calculated to produce a magnetic field strength in the range between 10⁻⁴ to 10⁻¹¹ Tesla. Although the specific signal tested was derived from a taxol sample, it will be appreciated that any taxane-like compound should generate a signal having the same mechanism of action in transduced form.

B. Time-Domain Signal Having an siRNA Mechanism of Action for Inhibition of mRNA Expression by a Selected Gene

In a second general embodiment, the invention provides a time-domain signal effective to produce a magnetic field capable of inhibiting mRNA expression of a selected gene in an in vitro cell culture assay, through a mechanism of action like that of siRNA targeting the selected gene. The signal was recorded from a sample of anti-GAPDH silencer RNA (siRNA) having the sequence:

5″ GGUCAUCCAUGACAACUUU 3′

3′ CCAGUAGGUACUGUUGAAA 5′, and was obtained commercially from Ambion as catalog number AM4624. A second, control scrambled sequence obtained from the same source has the sequence:

5′ AGUACUGCUUACGAUACGTT 3′
3′ TTUCAUGACGAAUGCUAUGCC 5′

For each siRNA, a solution of the siRNA, at a concentration of 20 uM was prepared in water. Signals were recorded with injected DC offset, at noise level settings between 10 and 241 mV and in increments of 1 mV. A total of 100 time-domain signals over this injected-noise level range were obtained, and these were analyzed by an enhanced autocorrelation algorithm detailed above, yielding 5 time-domain taxol signals for further in vitro testing. One of these, designated M23, was among the most effective of the 5 signals in the in vitro transduction studies described below.

The 10 mV offset anti-GAPDH siRNA signals from above was tested for its ability to generate a magnetic filed effective to inhibit GAPDH mRNA and protein in A549 cells (Human lung carcinoma, maintained according to the American Type Culture Collection.) Cells where plated at 82,000 cells/well (6-well format) the day before (24 hr recovery after plating) and placed in the transduction system described above with respect to FIG. 7B. The drug signal targeting GAPDH mRNA (experimental group) or the scrambled GAPDH signal (control group) was delivered for 72 h. The transduction signal applied to the coils was calculated to produce that cycles between 10⁻⁴ and 10⁻¹¹ Tesla, over 60 equal increments (approximately 10 mAmp change/increment), where each increment was pulsed for 1 second, followed by a brief cessation, giving a total cycle time of about 1 minute, and these cycles were repeated for the duration of the study. In this case three days. After 72 h, the cells where visually inspected under the microscope, and if found healthy, the procedure was continued by washing the cells in 1 ml PBS, at room temperature, treating with 0.3 ml Trypsin for 15 minutes also at room temperature, then resuspending in 1 ml complete grow medium.

The resuspended cells were counted 20,000 cells from each well were prepared for GAPDH reporter assay in an Eppendorf tube. The volume was adjusted to a total volume of 160 micro litters (μl) by adding the corresponding of complete grow medium. The KDalert GAPDH Assay Kit (Ambion, Tex.) catalog number 1639, was used according to manufactures protocol. Briefly, the cells where lysed in KDalert Lysis buffer for 1 h at 4 C and vortexed to mix the reagents. In a fresh Eppendorf tube, 10 μl cell lysate plus 80 ul KDalert reaction buffer was added according to manufacture's protocol. Each sample was read at t=0 and the again at t=6 min in a fluorometer (TD-700, Turner Designs, CA). The increase in fluorescence over time (GAPDH activity) is directly correlated to the amount of GAPDH protein in the sample.

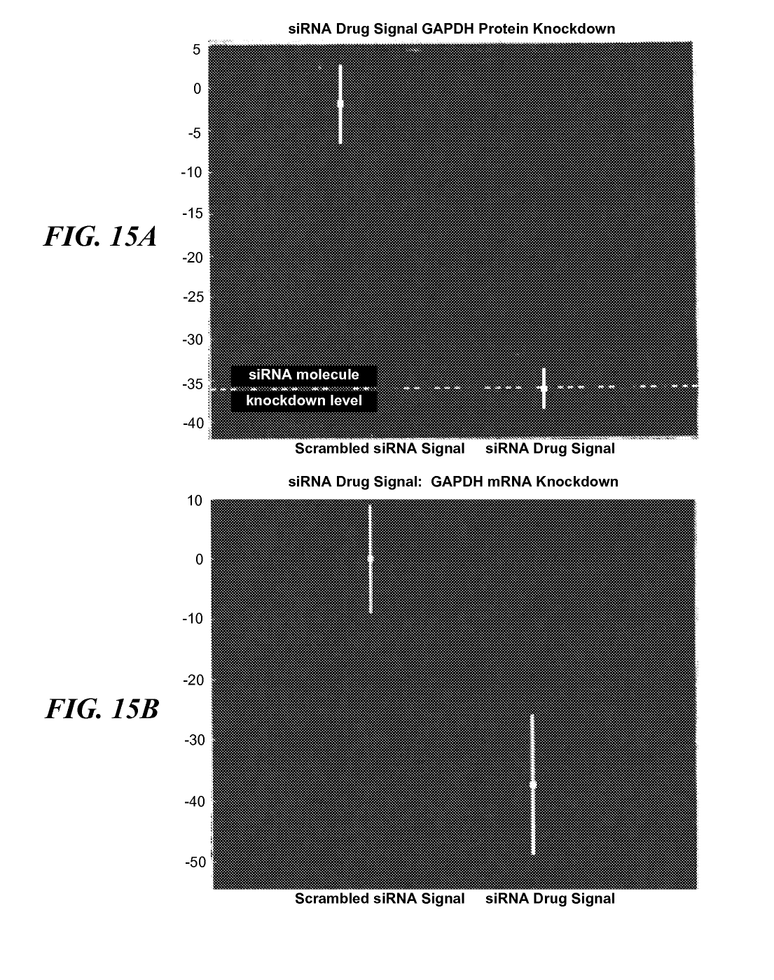

Levels of measured GAPDH mRNA in cells exposed to the scrambled control and GAPDH siRNA signal are plotted in FIG. 15B, plotted as a function of change in measured GAPDH relative to the scramble control, where the line for control and GAPDH signals represents the total spread in values among 6 and 12 cell samples. As seen, the magnetic file produced by the GAPDH siRNA signal was effective to inhibit GAPDH expression by greater than 35%.

To demonstrate that the inhibition in GAPDH expression was due to an inhibition of GAPDH expression, consistent with the proposed mechanism of action of the time-domain signal, GAPDH mRNA levels in the two groups of cells were assayed, employing a standard PCR assay using GAPDH-sequence specific primers. The results, plotted in FIG. 15B, are consistent with the protein-expression data: the siRNA GAPDH signal was effective in inhibiting GAPDH mRNA by about 35% relative to the scrambled-sequence control signal.

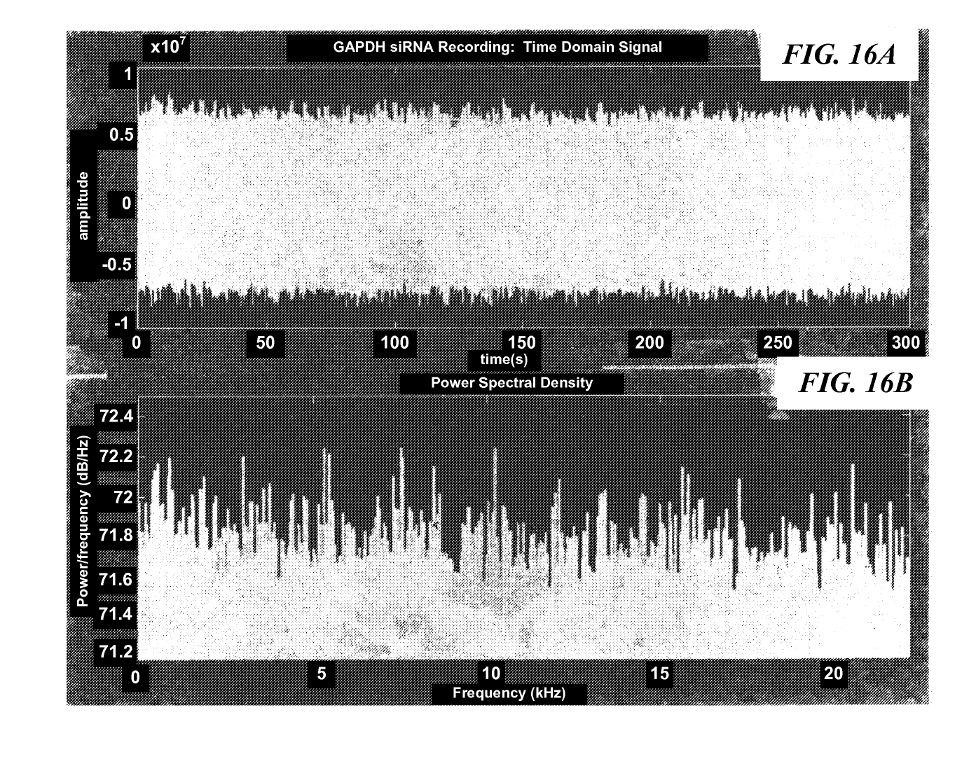

A 60-second portion of the GAPDH siRNA time domain signal used in the studies above is shown in FIG. 16A, and the signals' power spectral density, generated by the enhanced autocorrelation method detailed in Section IIIB above, is seen in FIG. 16B. As above, the second figure illustrates spectral features associated with the time-domain signal, and illustrates how the signal may be scored.

As discussed above, the signal is stored on a compact disc or any other suitable storage media for analog or digital signals and supplied to the transduction system during a signal transduction operation, for effecting a microtubule stabilization response in the target system. The signal carried on the compact disc is representative, more generally, of a tangible data storage medium having stored thereon, a low-frequency time domain signal effective to produce a magnetic field capable of inhibiting mRNA of a selected gene, through an siRNA mechanism of action, when the signal is supplied to electromagnetic transduction coil(s) at a signal current calculated to produce a magnetic field strength in the range between 10⁻⁴ to 10⁻¹¹ Tesla.

It will be appreciated from the two examples above how time-domain signals capable of producing one of a variety of selected mechanism-of-action effects in an in vitro and in vivo system can be generated. For example, a number of drugs that act to interfere with tubulin assembly, stability, and or disassembly, such as colchicine and the vinca alkaloids, can be used in accordance with the procedures above, to identify time-domain signals having the mechanism of action of the compound itself.

As another example, a large number of drugs function through their ability to bind to specific cell receptors, e.g., G protein receptors. For purposes of in vitro testing, there are many different mammalian cells, often with a genetically altered genome designed for allowing detection of agent binding to the target receptor, e.g., through the expression of a recombinant fluorescent protein, that can be cultured under conditions that would allow for the effects of signal transduction of the cells to be observed. Thus, in this treatment model, the transducing agent is the receptor-binding molecule, the in vitro system is a cell-culture system that is responsive to agent binding to produce a detectable cellular response, and the mammalian system is a mammalian subject having a disease state that is amenable to treatment by the binding agent.

Similarly, a number of drugs function through their ability to inhibit the activity of a soluble or membrane-associated enzyme. For in vitro testing, the target enzyme is likely to be adaptable to an in vitro enzyme reaction assay in which a drug effect on the activity of the enzyme can be detected, e.g., colorometrically, as an increase or decrease in enzyme activity with respect to a detectable substrate. Thus, in this treatment model, the transducing agent is the enzyme binding agent, the in vitro system is an enzyme assay reaction which is responsive to agent to produce a detectable change in enzyme kinetics, and the mammalian system is a mammalian subject having a disease state normally treated by the binding agent.

### VI. Treating Cancer In Vivo by Transduction with Time-Domain Signals Effective to Stabilize Microtubule Formation

Based on the demonstrated ability of the M2(3) signal to stabilize microtubule formation in a tubulin suspension assay and in cell culture, the same signal was tested for its ability to inhibit a tumor whose cells are known to be inhibit by a taxane compound such as taxol. In this study, two groups of 10 mice each were each injected in the right frontal lobe with 5×10⁻⁵ U87 glioblastoma cells, and treatment with M2(3) signals was begun one day later. The transduction device used in this study was a 2-ft diameter right-angle cylinder with coil windings, such as described with respect to FIG. 7C. These cylinders accommodate a standard mouse or rat cage so that mice are constantly exposed to the playback of signals.

During the treatment, all ten mice in each group are housed in one cage and kept within the area of the central cylindrical cavity of the large transduction coil under continuous playback, while they are fed and watered. This results in a continuous exposure duty time of about 90-95% of the study duration of 60 days. The treatment involved either no signal (control) or the M2(3) signal, applied to the coil by continuously sweeping the signal over an incremented magnetic field strength between 10⁻⁴ and 10⁻¹¹ Tesla, in 60 1 second increments and 1 minute cycles, as above, over the 23 day treatment period in the study. That is, each signal is played continuously to each of ten animals in a group, by sweeping the signal over a selected magnetic-field range, with only occasional interruption for cleaning and feeding.

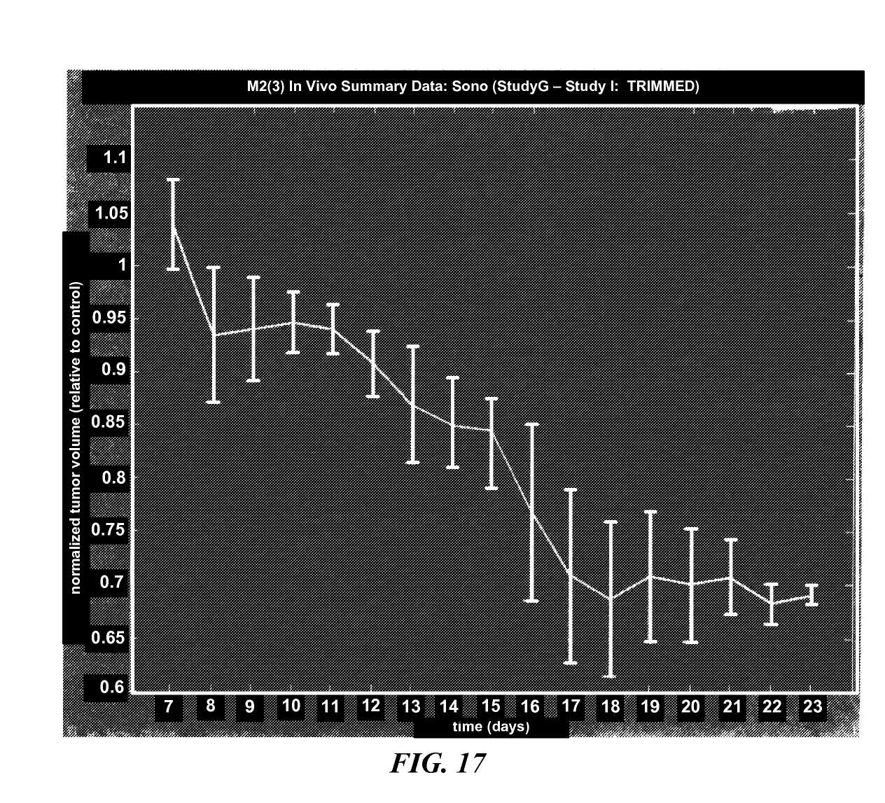

At day 23, the animals were sacrificed and their tumor removed and weighed. The results of the study, plotted as tumor number of animals surviving in each group over the 23 day period, are plotted as normalized tumor volume relative to control, are shown in FIG. 17. (Control animal tumor weights were averaged; the bars in the graph represent variation in treated-animal tumors weights relative to the average control-animal tumor weight). As seen, exposure to the tubulin-stabilizing magnetic fields, over a 23 day period, reduced tumor volume on average about 30%. A similar result was seen in tumor-bearing animals treated with taxol compound over the same period.

The study above points to several important therapeutic advantages that signal therapy may offer over conventional drug therapy. One important advantage is in “drug delivery.” As is well known, treatment of a variety of central nervous system (CNS) conditions, such as brain tumors, is not practical for a variety of anti-tumor compounds, including taxane compounds, because of the poor delivery across the blood brain barrier. The present approach avoids this limitation, of course, because the magnetic fields will be distributed throughout the biological system, irrespective of drug-physical barriers. In one aspect, therefore, the invention treating a CNS condition in a subject that would otherwise be susceptible to a selected drug-treatment, but for the presence of the blood brain barrier, where the treatment field is one having the same therapeutic mechanism of action as the otherwise effective drug. In particular, the invention contemplates treating a brain tumor, such as a glioblastoma, by exposure to a magnetic field capable of stabilizing microtubule formation in a tublulin suspension in vitro.

Another potential advantage of signal therapy over the conventional drug therapy is the potential for reduced side effects. Based on preliminary observations of animals undergoing signal therapy, there was little or no effect on activity level or behavior noticed in the treatment group relative to control animals, whereas animal being treated with taxol showed the expected signs of lethargy and loss of appetite. There are a number of possible explanations for reduced side effects, including the absence of peak drug concentrations associated with drug therapy, the fact that no toxic metabolic byproducts are being formed, the fact that drug is not accumulating within certain compartments of the body, and the possibility that the signal itself does not affect targets in the body that are sensitive to drug compounds.

The above detailed description of embodiments of the invention is not intended to be exhaustive or to limit the invention to the precise form disclosed above. While specific embodiments of, and examples for, the invention are described above for illustrative purposes, various equivalent modifications are possible within the scope of the invention, as those skilled in the relevant art will recognize. For example, while processes or blocks are presented in a given order, alternative embodiments may perform routines having steps, or employ systems having blocks, in a different order, and some processes or blocks may be deleted, moved, added, subdivided, combined, and/or modified. Each of these processes or blocks may be implemented in a variety of different ways. Also, while processes or blocks are at times shown as being performed in series, these processes or blocks may instead be performed in parallel, or may be performed at different times.

The teachings of the invention provided herein can be applied to other systems, not necessarily the system described above. The elements and acts of the various embodiments described above can be combined to provide further embodiments.

All of the above patents and applications and other references, including any that may be listed in accompanying filing papers, are incorporated herein by reference. Aspects of the invention can be modified, if necessary, to employ the systems, functions, and concepts of the various references described above to provide yet further embodiments of the invention.

It is claimed:

1. A tangible data storage medium having stored thereon, a low-frequency time domain signal effective to produce a magnetic field capable of stabilizing microtubule formation in an in vitro tubulin assay containing a suspension of tubulin, where the signal is supplied to electromagnetic transduction coil(s) at a signal current calculated to produce a magnetic field strength in the range between 10⁻⁴ to 10⁻¹¹ Tesla, and where the degree of stabilization of microtubule formation in the assay produced in the presence of the magnetic field is substantially greater than that observed in the absence of the field.

2. The storage medium of claim 1, wherein the signal is produced by the steps of:
(a) placing in a sample container having both magnetic and electromagnetic shielding, a sample of a taxane-like compound known to stabilize microtubule formation in such a tubulin sample, wherein the sample acts as a signal source for low-frequency molecular signals; and wherein the magnetic shielding is external to a cryogenic container;
(b) recording a plurality of low-frequency, time-domain signals composed of sample source radiation in the cryogenic container,
(c) scoring the signals produced in step (b) by one of (i) the peak areas values above a predetermined value as determined from an enhanced autocorrelation of the signal, and (ii) a histogram of the power spectrum of the signal, determined by spectral analysis, and
(d) identifying from among the signals having the highest score or scores from step (c) one or more signals that are effective in stabilizing microtubule formation in an in vitro tubulin assay, when the tubulin sample is exposed to a magnetic field produced by supplying the signal to electromagnetic transducer coil(s) at a signal current calculated to produce a magnetic field strength in the range between 10⁻⁴ to 10⁻¹¹ Tesla.

3. The storage medium of claim 1, wherein step (b) in producing the signal by recording a plurality of low-frequency, time-domain signals composed of sample source radiation in the cryogenic container includes recording the signals at each of a plurality of different stimulus magnetic field conditions selected from the group consisting of: (i) white noise, injected at voltage level calculated to produce a selected-strength magnetic field at the sample of between 0 and 1 G (Gauss), and/or (ii) a DC offset, injected at voltage level calculated to produce a selected-strength magnetic field at the sample of between 0 and 1 G.

4. The storage medium of claim 3, wherein step (b) used in producing the signal further includes scoring the signal by one of: (i) the peak areas values above a predetermined value as determined from an enhanced autocorrelation of the signal, and (ii) a histogram of the power spectrum of the signal, determined by spectral analysis, and said scoring in step (c) is carried out for signals produced at each of the different injected magnetic field conditions.

5. The storage medium of claim 1, wherein step (a) used in producing the signal includes preparing a sample of a taxane compound in an aqueous medium having a physiological salt concentration.

6. The storage medium of claim 5, wherein the taxane compound is taxol.

7. The storage medium of claim 1, wherein the time-domain signal is effective to stabilize microtubule formation in the in vitro tubulin assay, when the tubulin suspension is exposed to a magnetic field produced by supplying the signal to electromagnetic transducer coil(s) at a signal current calculated to produce an incremented magnetic field which is cycled in a range between 10⁻⁴ to 10⁻¹¹ Tesla.

8. A tangible data storage medium having stored thereon, a low-frequency time domain signal effective to produce a magnetic field capable of inhibiting mRNA expression of a selected gene in an in vitro cell culture assay, where the signal is supplied to electromagnetic transduction coil(s) at a signal current calculated to produce a magnetic field strength in the range between 10⁻⁴ to 10⁻¹¹ Tesla, and where the degree of inhibition of mRNA transcription in the assay in the presence of the magnetic field is substantially greater than that observed in the absence of the field.

9. The storage medium of claim 8, wherein the signal is produced by the steps of:
(a) placing in a sample container having both magnetic and electromagnetic shielding, a sample of an siRNA compound known to inhibit mRNA expression of the selected gene in an in vitro assay in which the cells are exposed to the compound, wherein the sample acts as a signal source for low-frequency molecular signals; and wherein the magnetic shielding is external to a cryogenic container;
(b) recording a plurality of low-frequency, time-domain signals composed of sample source radiation in the cryogenic container,
(c) scoring the signals produced in step (b) by one of (i) the peak areas values above a predetermined value as determined from an enhanced autocorrelation of the signal, and (ii) a histogram of the power spectrum of the signal, determined by spectral analysis, and
(d) identifying from among the signals having the highest score or scores from step (c) one or more signals that are most effective in inhibiting mRNA expression of the selected gene, when an in vitro cell culture containing such cells is exposed to a magnetic field produced by supplying the signal to electromagnetic transducer coil(s) at a signal current calculated to produce a magnetic field in the range between 10⁻⁴ to 10⁻¹¹ Tesla.

10. The storage medium of claim 9, wherein step (b) in producing the signal by recording a plurality of low-frequency, time-domain signals composed of sample source radiation in the cryogenic container includes recording the signals at each of a plurality of different stimulus magnetic field conditions selected from the group consisting of: (i) white noise, injected at voltage level calculated to produce a selected-strength magnetic field at the sample of between 0 and 1 G (Gauss), and/or (ii) a DC offset, injected at voltage level calculated to produce a selected-strength magnetic field at the sample of between 0 and 1 G.

11. The storage medium of claim 10, wherein step (b) used in producing the signal further includes scoring the signal by one of: (i) the peak areas values above a predetermined value as determined from an enhanced autocorrelation of the signal, and (ii) a histogram of the power spectrum of the signal, determined by spectral analysis, and said scoring in step (c) is carried out for signals produced at each of the different injected magnetic field conditions.

12. The storage medium of claim 8, wherein step (a) used in producing the signal includes preparing an anti-GADPH siRNA sample in an aqueous medium having a physiological salt concentration.

13. The storage medium of claim 12, wherein the anti-GADPH is a double-stranded RNA having the sequence identified by SEQ ID NO: 1.

14. The storage medium of claim 8, wherein the time-domain signal is effective to inhibit expression of GADPH mRNA in the in vitro assay in which 549 lung carcinoma cells are exposed to magnetic field produced by supplying the signal to electromagnetic transducer coil(s) at a signal current calculated to produce an incremented magnetic field which is cycled in a range between 10⁻⁴ to 10⁻¹¹ Tesla.

15. In a method for producing an agent-like response in a system, by exposing the system to a magnetic field produced by one or more electromagnetic transducer coils to which is supplied a selected low-frequency time-domain signal over a given exposure period, an improvement comprising
adjusting the magnetic field to which the subject is exposed by applying to the transducer coils, a signal current calculated to produce a magnetic field in the range between 10⁻⁴ to 10⁻¹¹ Tesla, where the magnetic field supplied to the subject is supplied in cycles of varying-field increments, over a selected signal-current range, where each signal-current increment in a cycle is applied in defined-duration pulses over the known given period.

16. The improvement of claim 15, for use in treating a subject having a tumor whose cells are inhibited in the presence of taxol, wherein the magnetic field to which the subject is exposed is produced by supplying to the one or more electromagnetic transduction coils, a low-frequency time domain signal effective to produce a magnetic field capable of stabilizing microtubule formation in an in vitro tubulin assay containing a suspension of tubulin, where the signal is supplied to electromagnetic transduction coil(s) at a signal current calculated to produce a magnetic field strength in the range between 10⁻⁴ to 10⁻¹¹ Tesla, and where the degree of stabilization of microtubule formation in the assay produced in the presence of the magnetic field is substantially greater than that observed in the absence of the field.

17. The improvement of claim 16, wherein the low-frequency time-domain signal is produced by the steps of:
(a) placing in a sample container having both magnetic and electromagnetic shielding, a sample of a taxane-like compound known to stabilize microtubule formation in such a tubulin sample, wherein the sample acts as a signal source for low-frequency molecular signals; and wherein the magnetic shielding is external to a cryogenic container;
(b) recording a plurality of low-frequency, time-domain signals composed of sample source radiation in the cryogenic container,
(c) scoring the signals produced in step (b) by one of (i) the peak areas values above a predetermined value as determined from an enhanced autocorrelation of the signal, and (ii) a histogram of the power spectrum of the signal, determined by spectral analysis, and
(d) identifying from among the signals having the highest score or scores from step (c) one or more signals that are most effective in stabilizing microtubule formation in an in vitro tubulin assay, when the tubulin sample is exposed to a magnetic field produced by supplying the signal to electromagnetic transducer coil(s) at a signal current calculated to produce a magnetic field in the range between 10⁻⁴ to 10⁻¹¹ Tesla.

18. The improvement in claim 16, wherein the subject is exposed to the magnetic field, either continuously or on a daily basis, at least over a three-week treatment period, and the method further includes measuring changes in the size of the tumor over the treatment period.

19. The improvement of claim 15, for use in treating in a subject whose cells are inhibited in GADPH protein and mRNA expression by the presence of an anti-GADPG siRNA compound, wherein the magnetic field to which the subject is exposed is produced by supplying to the one or more electromagnetic transduction coils, a low-frequency time domain signal effective to produce an siRNA-specific inhibition of GADPH protein or GADPH mRNA, relative to that observed for a signal derived under identical conditions from a scrambled-sequence siRNA control, in an in vitro siRNA assay in which 549 lung carcinoma cells are exposed to magnetic field produced by supplying the signal to electromagnetic transducer coil(s) at a signal current calculated to produce a selected-strength magnetic field in the range between 10⁻⁴ to 10⁻¹¹ Tesla.

20. The improvement of claim 19, wherein the signal is produced by the steps of:
(a) placing in a sample container having both magnetic and electromagnetic shielding, a sample of an siRNA compound known to inhibit GADPH protein or GADPH mRNA expression in an in vitro assay in which 549 lung carcinoma cells are exposed to the compound, wherein the sample acts as a signal source for low-frequency molecular signals; and wherein the magnetic shielding is external to a cryogenic container;
(b) recording a plurality of low-frequency, time-domain signals composed of sample source radiation in the cryogenic container,
(c) scoring the signals produced in step (b) by one of (i) the peak areas values above a predetermined value as determined from an enhanced autocorrelation of the signal, and (ii) a histogram of the power spectrum of the signal, determined by spectral analysis, and
(d) identifying from among the signals having the highest score or scores from step (c) one or more signals that are most effective in stabilizing microtubule formation in an in vitro tubulin assay, when the tubulin sample is exposed to a magnetic field produced by supplying the signal to electromagnetic transducer coil(s) at a signal current calculated to produce a magnetic field in the range between 10⁻⁴ to 10⁻¹¹ Tesla.

21. A method of treating a subject having a condition that is responsive to a therapeutic agent capable of producing an observable agent-specific effect in an in vitro cell-culture or cell-free system, comprising
(a) placing the subject system within the interior region of one or more electromagnetic transducer coils,
(b) supplying to the transducer coils, a low-frequency time-domain signal to produce a magnetic field that is effective, when supplied to the in vitro system under identical conditions, to produce the agent-specific effect, at a signal current calculated to produce a selected-strength magnetic field at the coils in the 10⁻⁴ to 10⁻¹¹ Tesla range, and
(c) exposing the subject to the magnetic field produced in step (b), over a time period sufficient to produce a measurable agent-specific response in the subject.

22. The method of claim 21, for use in treating in a subject, a tumor whose cells are inhibited in the presence of taxol, and the low-frequency signal to which the subject is exposed is effective to produce a magnetic field capable of stabilizing microtubule formation in an in vitro tubulin assay containing a suspension of tubulin, where the signal is supplied to electromagnetic transduction coil(s) at a signal current calculated to produce a magnetic field strength in the range between 10⁻⁴ to 10⁻⁴¹ Tesla.

23. The method of claim 21, for use in treating a condition of the CNS that would be responsive to the therapeutic agent, but for the presence of the blood brain barrier, wherein exposing step (c) includes exposing the region of the CNS having such condition to the magnetic field.

24. The method of claim 23, for use in treating in a subject, a CNS tumor whose cells are inhibited in the presence of taxol, and the low-frequency signal to which the subject is exposed is effective to produce a magnetic field capable of stabilizing microtubule formation in an in vitro tubulin assay containing a suspension of tubulin, where the signal is supplied to electromagnetic transduction coil(s) at a signal current calculated to produce a magnetic field strength in the range between 10⁻⁴ to 10⁻¹¹ Tesla.
